SEQUENCE LISTING (169 sequences)

SEQ ID NO: 1 - protein, 757 aa, unidentified
  source 1..757: note = Huge Phage
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 2 - protein, 707 aa, unidentified
  source 1..707: note = Huge phage
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 3 - protein, 766 aa, unidentified
  source 1..766: note = Huge phage
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 4 - protein, 740 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKAGSSGEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 5 - protein, 773 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSKRPAATKKAGQAKKKK

SEQ ID NO: 6 - protein, 787 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKAGSSGEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSKRPAATKKAGQAKKKKGSDYKDHDGDYKDHDIDYKDDDDKPKKKRKV

SEQ ID NO: 7 - protein, 16 aa, synthetic construct
KRPAATKKAGQAKKKK

SEQ ID NO: 8 - protein, 31 aa, synthetic construct
GSDYKDHDGDYKDHDIDYKDDDDKPKKKRKV

SEQ ID NO: 9 - DNA, 2412 bp, synthetic construct
  source 1..2412: mol_type = other DNA
atgcctaagcctgctgttgagagcgagttctctaaggtgctcaagaagcactttcctggcgagaggttccgtagctcttacatgaagagaggtgggaagatccttgctgctcaaggtgaagaagctgtggttgcttacctccagggaaagtctgaagaggaacctcctaacttccagcctcctgctaagtgccatgtggtgactaagtctagggatttcgctgagtggccaatcatgaaggcttcagaggctatccagcgttacatctacgctttgtctaccaccgagagagctgcttgtaagccgggaaaatcttctgagtctcacgctgcttggtttgctgctaccggtgtgtctaatcacggatactctcatgtgcaggggctcaacctcatcttcgatcatactctcggaaggtacgacggggttctcaagaaagttcagctccgtaacgagaaggctagggctagacttgagagcatcaacgcttcaagagctgatgagggactccctgagattaaggcagaggaagaggaagttgccaccaacgagactggacatcttcttcaacctccgggaatcaacccgagcttctacgtgtaccaaaccatctctccacaggcttacagacctagggatgagatcgttcttccacctgagtacgctggttacgtgagagatcctaacgctcctattcctctcggagtggtgagaaacagatgcgacatccaaaagggatgccctggttacattcctgagtggcaaagagaagctgggaccgctatttctcctaagactggaaaggctgtgaccgtgcctggactctctccaaagaaaaacaagaggatgcgtaggtactggcgtagcgagaaagaaaaggctcaggatgctctcctcgtgaccgttagaattggaaccgattgggttgtgatcgacgtgagaggacttctcagaaacgctaggtggcgtactatcgctcctaaggatattagcctcaacgccctccttgatctcttcactggtgatcctgttatcgacgtgcgtaggaacatcgtgaccttcacttacactctcgatgcttgcggaacctacgctagaaagtggactcttaaggggaagcagactaaggcgactctcgataagttgactgctactcagactgtggctctcgtggctatcgatcttggacagactaaccctatcagcgctggaatctctagggtgacacaagaaaacggtgctctccagtgtgagcctctcgataggtttactctccctgatgatctcctcaaggacatcagcgcttacaggatcgcttgggatcgtaacgaggaagagttgagggcaagatctgttgaggctttgcctgaggctcaacaggctgaagtgagagctttggacggtgtctctaaagagactgctagaactcagctctgcgctgatttcggactcgatcctaagagactcccttgggacaagatgtcctctaacaccacctttatcagcgaggccctcctctctaactctgtgtctcgtgatcaggtgttcttcacccctgctcctaagaagggtgctaagaagaaggctcctgttgaggtcatgaggaaggatagaacttgggctagagcctacaagccaagactctctgtggaagcccagaagttgaagaacgaagctctttgggctctcaagaggacctctcctgagtaccttaagctcagcaggcgtaaagaggaactctgcaggcgtagcatcaactacgtgatcgaaaagaccagacgtaggacccagtgccagatcgttatcccagttatcgaggacctcaacgtccgtttctttcacggatctggtaagaggcttcctggctgggataatttcttcaccgccaagaaagagaaccgttggttcatccagggactccacaaggctttctctgatctcagaacccaccgttccttctacgttttcgaggttaggcctgagcgaacctctatcacttgtcctaagtgtggacattgcgaggtcggaaacagagatggtgaggctttccagtgtctctcttgcggaaagacttgcaacgctgatctcgatgtggctacccacaacctcactcaagttgctctcactggtaagaccatgccgaagagggaagaacctagagatgctcagggaactgctcctgctagaaagaccaagaaggccagcaagtctaaggctcctcctgctgagagagaggatcaaactcctgctcaagagccttctcagacctctaaaaggccggcggccacgaaaaaggccggccaggcaaaaaagaaaaagggatccgactataaggaccacgacggagactacaaggatcatgatattgattacaaagacgatgacgataagccaaagaagaagcggaaggtc

SEQ ID NO: 10 - DNA, 2412 bp, synthetic construct; identical to SEQ ID NO: 9
  source 1..2412: mol_type = other DNA

SEQ ID NO: 11 - DNA, 2364 bp, synthetic construct
  source 1..2364: mol_type = other DNA
atgcctaagcctgctgttgagagcgagttctctaaggtgctcaagaagcactttcctggcgagaggttccgtagctcttacatgaagagaggtgggaagatccttgctgctcaaggtgaagaagctgtggttgcttacctccagggaaagtctgaagaggaacctcctaacttccagcctcctgctaagtgccatgtggtgactaagtctagggatttcgctgagtggccaatcatgaaggcttcagaggctatccagcgttacatctacgctttgtctaccaccgagagagctgcttgtaagccgggaaaatcttctgagtctcacgctgcttggtttgctgctaccggtgtgtctaatcacggatactctcatgtgcaggggctcaacctcatcttcgatcatactctcggaaggtacgacggggttctcaagaaagttcagctccgtaacgagaaggctggaagttctggcgaggaagaggaagttgccaccaacgagactggacatcttcttcaacctccgggaatcaacccgagcttctacgtgtaccaaaccatctctccacaggcttacagacctagggatgagatcgttcttccacctgagtacgctggttacgtgagagatcctaacgctcctattcctctcggagtggtgagaaacagatgcgacatccaaaagggatgccctggttacattcctgagtggcaaagagaagctgggaccgctatttctcctaagactggaaaggctgtgaccgtgcctggactctctccaaagaaaaacaagaggatgcgtaggtactggcgtagcgagaaagaaaaggctcaggatgctctcctcgtgaccgttagaattggaaccgattgggttgtgatcgacgtgagaggacttctcagaaacgctaggtggcgtactatcgctcctaaggatattagcctcaacgccctccttgatctcttcactggtgatcctgttatcgacgtgcgtaggaacatcgtgaccttcacttacactctcgatgcttgcggaacctacgctagaaagtggactcttaaggggaagcagactaaggcgactctcgataagttgactgctactcagactgtggctctcgtggctatcgatcttggacagactaaccctatcagcgctggaatctctagggtgacacaagaaaacggtgctctccagtgtgagcctctcgataggtttactctccctgatgatctcctcaaggacatcagcgcttacaggatcgcttgggatcgtaacgaggaagagttgagggcaagatctgttgaggctttgcctgaggctcaacaggctgaagtgagagctttggacggtgtctctaaagagactgctagaactcagctctgcgctgatttcggactcgatcctaagagactcccttgggacaagatgtcctctaacaccacctttatcagcgaggccctcctctctaactctgtgtctcgtgatcaggtgttcttcacccctgctcctaagaagggtgctaagaagaaggctcctgttgaggtcatgaggaaggatagaacttgggctagagcctacaagccaagactctctgtggaagcccagaagttgaagaacgaagctctttgggctctcaagaggacctctcctgagtaccttaagctcagcaggcgtaaagaggaactctgcaggcgtagcatcaactacgtgatcgaaaagaccagacgtaggacccagtgccagatcgttatcccagttatcgaggacctcaacgtccgtttctttcacggatctggtaagaggcttcctggctgggataatttcttcaccgccaagaaagagaaccgttggttcatccagggactccacaaggctttctctgatctcagaacccaccgttccttctacgttttcgaggttaggcctgagcgaacctctatcacttgtcctaagtgtggacattgcgaggtcggaaacagagatggtgaggctttccagtgtctctcttgcggaaagacttgcaacgctgatctcgatgtggctacccacaacctcactcaagttgctctcactggtaagaccatgccgaagagggaagaacctagagatgctcagggaactgctcctgctagaaagaccaagaaggccagcaagtctaaggctcctcctgctgagagagaggatcaaactcctgctcaagagccttctcagacctctaaaaggccggcggccacgaaaaaggccggccaggcaaaaaagaaaaagggatccgactataaggaccacgacggagactacaaggatcatgatattgattacaaagacgatgacgataagccaaagaagaagcggaaggtctaa

SEQ ID NO: 12 - DNA, 36 bp, synthetic construct
  source 1..36: mol_type = other DNA
gtcggaacgctcaacgattgcccctcacgaggggac

SEQ ID NO: 13 - DNA, 36 bp, synthetic construct
  source 1..36: mol_type = other DNA
ggagagatctcaaacgattgctcgattagtcgagac

SEQ ID NO: 14 - DNA, 36 bp, synthetic construct
  source 1..36: mol_type = other DNA
accaaaacgactattgattgcccagtacgctgggac

SEQ ID NO: 15 - DNA, 59 bp, synthetic construct
  source 1..59: mol_type = other DNA
  misc_feature 1..10
cgagagtgtcgttcgtaaaaaaaagagaccctctctcggtctccgtcccctcgtgaggg

SEQ ID NO: 16 - DNA, 59 bp, synthetic construct
  source 1..59: mol_type = other DNA
ccctcacgaggggacggagaccgagagagggtctcttttttttacgaacgacactctcg

SEQ ID NO: 17 - DNA, 66 bp, synthetic construct
  source 1..66: mol_type = other DNA
tcctccagagactttccgcttcttctttggggctttatgattcatatgtatatctccttcttaaag

SEQ ID NO: 18 - DNA, 78 bp, synthetic construct
  source 1..78: mol_type = other DNA
tctggcggctcaaaaagaaccgccgacggcagcgaattcgagcccaagaagaagaggaaagtcctcgagcaccaccac

SEQ ID NO: 19 - DNA, 66 bp, synthetic construct; identical to SEQ ID NO: 17
  source 1..66: mol_type = other DNA

SEQ ID NO: 20 - DNA, 78 bp, synthetic construct; identical to SEQ ID NO: 18
  source 1..78: mol_type = other DNA

SEQ ID NO: 21 - DNA, 22 bp, synthetic construct
  source 1..22: mol_type = other DNA
ggaccagttgacaatccagcca

SEQ ID NO: 22 - DNA, 22 bp, synthetic construct
  source 1..22: mol_type = other DNA
acaatggctggattgtcaactg

SEQ ID NO: 23 - DNA, 20 bp, synthetic construct
  source 1..20: mol_type = other DNA
ggaccagttgacaatccagc

SEQ ID NO: 24 - DNA, 20 bp, synthetic construct
  source 1..20: mol_type = other DNA
acaagctggattgtcaactg

SEQ ID NO: 25 - DNA, 19 bp, synthetic construct
  source 1..19: mol_type = other DNA
ggaccagttgacaatccag

SEQ ID NO: 26 - DNA, 19 bp, synthetic construct
  source 1..19: mol_type = other DNA
acaactggattgtcaactg

SEQ ID NO: 27 - DNA, 61 bp, synthetic construct
  source 1..61: mol_type = other DNA
tcgtcggcagcgtcagatgtgtataagagacagggaaaagaatcatatggtcatcaattcg

SEQ ID NO: 28 - DNA, 62 bp, synthetic construct
  source 1..62: mol_type = other DNA
gtctcgtgggctcggagatgtgtataagagacagcggacttgaattaaaggggctatagtag

SEQ ID NO: 29 - DNA, 60 bp, synthetic construct
  source 1..60: mol_type = other DNA
tcgtcggcagcgtcagatgtgtataagagacaggcaaataattaaagttgttgctgttgg

SEQ ID NO: 30 - DNA, 55 bp, synthetic construct
  source 1..55: mol_type = other DNA
gtctcgtgggctcggagatgtgtataagagacagtagcctacttgcctgcttttc

SEQ ID NO: 31 - DNA, 21 bp, synthetic construct
  source 1..21: mol_type = other DNA
ggacccaacgaagacctcgag

SEQ ID NO: 32 - DNA, 21 bp, synthetic construct
  source 1..21: mol_type = other DNA
acaactcgaggtcttcgttgg

SEQ ID NO: 33 - DNA, 21 bp, synthetic construct
  source 1..21: mol_type = other DNA
ggacctcacgcccaactaaac

SEQ ID NO: 34 - DNA, 21 bp, synthetic construct
  source 1..21: mol_type = other DNA
acaagtttagttgggcgtgag

SEQ ID NO: 35 - DNA, 21 bp, synthetic construct
  source 1..21: mol_type = other DNA
ggacgtatttgcacccgcaga

SEQ ID NO: 36 - DNA, 21 bp, synthetic construct
  source 1..21: mol_type = other DNA
acaatctgcgggtgcaaatac

SEQ ID NO: 37 - DNA, 20 bp, synthetic construct
  source 1..20: mol_type = other DNA
ggactccaagaccagagcta

SEQ ID NO: 38 - DNA, 20 bp, synthetic construct
  source 1..20: mol_type = other DNA
acaatagctctggtcttgga

SEQ ID NO: 39 - DNA, 21 bp, synthetic construct
  source 1..21: mol_type = other DNA
ggaccctcagtgtgtacgctg

SEQ ID NO: 40 - DNA, 20 bp, synthetic construct
  source 1..20: mol_type = other DNA
acaacagcgtacacactgag

SEQ ID NO: 41 - DNA, 21 bp, synthetic construct
  source 1..21: mol_type = other DNA
ggaccaagcaaacatcttgac

SEQ ID NO: 42 - DNA, 21 bp, synthetic construct
  source 1..21: mol_type = other DNA
acaagtcaagatgtttgcttg

SEQ ID NO: 43 - DNA, 21 bp, synthetic construct
  source 1..21: mol_type = other DNA
ggaccatgccgattgtggaac

SEQ ID NO: 44 - DNA, 21 bp, synthetic construct
  source 1..21: mol_type = other DNA
acaagttccacaatcggcatg

SEQ ID NO: 45 - DNA, 21 bp, synthetic construct
  source 1..21: mol_type = other DNA
ggacctctttccccatgaagt

SEQ ID NO: 46 - DNA, 21 bp, synthetic construct
  source 1..21: mol_type = other DNA
acaaacttcatggggaaagag

SEQ ID NO: 47 - DNA, 21 bp, synthetic construct
  source 1..21: mol_type = other DNA
ggacaataggaatgtgctgac

SEQ ID NO: 48 - DNA, 21 bp, synthetic construct
  source 1..21: mol_type = other DNA
acaagtcagcacattcctatt

SEQ ID NO: 49 - DNA, 21 bp, synthetic construct
  source 1..21: mol_type = other DNA
ggaccaaagaacccgtgtctt

SEQ ID NO: 50 - DNA, 21 bp, synthetic construct
  source 1..21: mol_type = other DNA
acaaaagacacgggttctttg

SEQ ID NO: 51 - DNA, 20 bp, synthetic construct
  source 1..20: mol_type = other DNA
ggacctgctctggccgttga

SEQ ID NO: 52 - DNA, 20 bp, synthetic construct
  source 1..20: mol_type = other DNA
acaatcaacggccagagcag

SEQ ID NO: 53 - DNA, 20 bp, synthetic construct
  source 1..20: mol_type = other DNA
ggacatccaaacagcccatc

SEQ ID NO: 54 - DNA, 20 bp, synthetic construct
  source 1..20: mol_type = other DNA
acaagatgggctgtttggat

SEQ ID NO: 55 - DNA, 21 bp, synthetic construct
  source 1..21: mol_type = other DNA
ggacctcagcatattatatgc

SEQ ID NO: 56 - DNA, 21 bp, synthetic construct
  source 1..21: mol_type = other DNA
acaagcatataatatgctgag

SEQ ID NO: 57 - DNA, 21 bp, synthetic construct
  source 1..21: mol_type = other DNA
ggactctgtggattcgaattt

SEQ ID NO: 58 - DNA, 21 bp, synthetic construct
  source 1..21: mol_type = other DNA
acaaaaattcgaatccacaga

SEQ ID NO: 59 - DNA, 21 bp, synthetic construct
  source 1..21: mol_type = other DNA
ggacatgagataagtctaggt

SEQ ID NO: 60 - DNA, 21 bp, synthetic construct
  source 1..21: mol_type = other DNA
acaaacctagacttatctcat

SEQ ID NO: 61 - DNA, 21 bp, synthetic construct
  source 1..21: mol_type = other DNA
ggacgaggatcaataggtgta

SEQ ID NO: 62 - DNA, 21 bp, synthetic construct
  source 1..21: mol_type = other DNA
acaatacacctattgatcctc

SEQ ID NO: 63 - DNA, 21 bp, synthetic construct
  source 1..21: mol_type = other DNA
ggacaagtttgagatgaggtc

SEQ ID NO: 64 - DNA, 21 bp, synthetic construct
  source 1..21: mol_type = other DNA
acaagacctcatctcaaactt

SEQ ID NO: 65 - DNA, 20 bp, synthetic construct
  source 1..20: mol_type = other DNA
ggacgccagctcgaggtcaa

SEQ ID NO: 66 - DNA, 20 bp, synthetic construct
  source 1..20: mol_type = other DNA
acaattgacctcgagctggc

SEQ ID NO: 67 - DNA, 20 bp, synthetic construct
  source 1..20: mol_type = other DNA
ggacgaaatccagcaaagca

SEQ ID NO: 68 - DNA, 20 bp, synthetic construct
  source 1..20: mol_type = other DNA
acaatgctttgctggatttc

SEQ ID NO: 69 - DNA, 21 bp, synthetic construct
  source 1..21: mol_type = other DNA
ggactggcattctggttgccc

SEQ ID NO: 70 - DNA, 21 bp, synthetic construct
  source 1..21: mol_type = other DNA
acaagggcaaccagaatgcca

SEQ ID NO: 71 - DNA, 21 bp, synthetic construct
  source 1..21: mol_type = other DNA
ggacaacctccgcttcccagt

SEQ ID NO: 72 - DNA, 21 bp, synthetic construct
  source 1..21: mol_type = other DNA
acaaactgggaagcggaggtt

SEQ ID NO: 73 - DNA, 56 bp, synthetic construct
  source 1..56: mol_type = other DNA
tcgtcggcagcgtcagatgtgtataagagacaggccccaaattggacttgtttctg

SEQ ID NO: 74 - DNA, 56 bp, synthetic construct
  source 1..56: mol_type = other DNA
gtctcgtgggctcggagatgtgtataagagacagccagcatcacactttcgcattc

SEQ ID NO: 75 - DNA, 56 bp, synthetic construct
  source 1..56: mol_type = other DNA
tcgtcggcagcgtcagatgtgtataagagacaggtgtgatgctggatttatgatcg

SEQ ID NO: 76 - DNA, 56 bp, synthetic construct
  source 1..56: mol_type = other DNA
gtctcgtgggctcggagatgtgtataagagacagctaatagaatgatcttccttcc

SEQ ID NO: 77 - DNA, 53 bp, synthetic construct
  source 1..53: mol_type = other DNA
tcgtcggcagcgtcagatgtgtataagagacaggggacctgacatatcggtgc

SEQ ID NO: 78 - DNA, 56 bp, synthetic construct
  source 1..56: mol_type = other DNA
gtctcgtgggctcggagatgtgtataagagacaggacacttccatcctcattcagc

SEQ ID NO: 79 - DNA, 56 bp, synthetic construct
  source 1..56: mol_type = other DNA
tcgtcggcagcgtcagatgtgtataagagacaggctacatgtcgattatgttcccc

SEQ ID NO: 80 - DNA, 56 bp, synthetic construct
  source 1..56: mol_type = other DNA
gtctcgtgggctcggagatgtgtataagagacaggcagcaactcacacacaaatcc

SEQ ID NO: 81 - DNA, 55 bp, synthetic construct
  source 1..55: mol_type = other DNA
tcgtcggcagcgtcagatgtgtataagagacaggggtcctctgatataactggtc

SEQ ID NO: 82 - DNA, 55 bp, synthetic construct
  source 1..55: mol_type = other DNA
gtctcgtgggctcggagatgtgtataagagacagctcgcatactacacaactatg

SEQ ID NO: 83 - DNA, 54 bp, synthetic construct
  source 1..54: mol_type = other DNA
tcgtcggcagcgtcagatgtgtataagagacaggccccaaattggacttgtttc

SEQ ID NO: 84 - DNA, 55 bp, synthetic construct
  source 1..55: mol_type = other DNA
gtctcgtgggctcggagatgtgtataagagacaggctcgtgatcataaattcagc

SEQ ID NO: 85 - DNA, 54 bp, synthetic construct
  source 1..54: mol_type = other DNA
tcgtcggcagcgtcagatgtgtataagagacagtgatcacgagcatatattctc

SEQ ID NO: 86 - DNA, 56 bp, synthetic construct; identical to SEQ ID NO: 76
  source 1..56: mol_type = other DNA

SEQ ID NO: 87 - DNA, 54 bp, synthetic construct
  source 1..54: mol_type = other DNA
tcgtcggcagcgtcagatgtgtataagagacaggacctgacatattggtgcagg

SEQ ID NO: 88 - DNA, 55 bp, synthetic construct
  source 1..55: mol_type = other DNA
gtctcgtgggctcggagatgtgtataagagacaggacacttccatcctcattcag

SEQ ID NO: 89 - DNA, 56 bp, synthetic construct
  source 1..56: mol_type = other DNA
tcgtcggcagcgtcagatgtgtataagagacagggcaccattgttggtggagaaag

SEQ ID NO: 90 - DNA, 55 bp, synthetic construct
  source 1..55: mol_type = other DNA
gtctcgtgggctcggagatgtgtataagagacaggcaggacacagatctagcagc

SEQ ID NO: 91 - DNA, 53 bp, synthetic construct
  source 1..53: mol_type = other DNA
tcgtcggcagcgtcagatgtgtataagagacaggcccactatatccgttcatg

SEQ ID NO: 92 - DNA, 55 bp, synthetic construct
  source 1..55: mol_type = other DNA
gtctcgtgggctcggagatgtgtataagagacaggcaaatgattactggccttgg

SEQ ID NO: 93 - DNA, 53 bp, synthetic construct
  source 1..53: mol_type = other DNA
tcgtcggcagcgtcagatgtgtataagagacaggtacttgagacagcatctgg

SEQ ID NO: 94 - DNA, 54 bp, synthetic construct
  source 1..54: mol_type = other DNA
gtctcgtgggctcggagatgtgtataagagacagggacatatatggtccatgcc

SEQ ID NO: 95 - DNA, 53 bp, synthetic construct
  source 1..53: mol_type = other DNA
tcgtcggcagcgtcagatgtgtataagagacaggacaagctcaagtccagcag

SEQ ID NO: 96 - DNA, 54 bp, synthetic construct
  source 1..54: mol_type = other DNA
gtctcgtgggctcggagatgtgtataagagacaggtggttcatgctgacctcag

SEQ ID NO: 97 - DNA, 53 bp, synthetic construct
  source 1..53: mol_type = other DNA
tcgtcggcagcgtcagatgtgtataagagacagcatctacagcctccagaatc

SEQ ID NO: 98 - DNA, 54 bp, synthetic construct
  source 1..54: mol_type = other DNA
gtctcgtgggctcggagatgtgtataagagacaggagcttctcagcttggggac

SEQ ID NO: 99 - DNA, 53 bp, synthetic construct
  source 1..53: mol_type = other DNA
tcgtcggcagcgtcagatgtgtataagagacaggctggttagctgagaatgcc

SEQ ID NO: 100 - DNA, 54 bp, synthetic construct
  source 1..54: mol_type = other DNA
gtctcgtgggctcggagatgtgtataagagacagctgtcttccaccgactgtgg

SEQ ID NO: 101 - DNA, 52 bp, synthetic construct
  source 1..52: mol_type = other DNA
tcgtcggcagcgtcagatgtgtataagagacagcttccaccgactgtggagc

SEQ ID NO: 102 - DNA, 54 bp, synthetic construct
  source 1..54: mol_type = other DNA
gtctcgtgggctcggagatgtgtataagagacaggctggttagctgagaatgcc

SEQ ID NO: 103 - DNA, 53 bp, synthetic construct
  source 1..53: mol_type = other DNA
tcgtcggcagcgtcagatgtgtataagagacaggacctcaggaataaggcaag

SEQ ID NO: 104 - DNA, 53 bp, synthetic construct
  source 1..53: mol_type = other DNA
gtctcgtgggctcggagatgtgtataagagacagggcgtaaaccaggaacatc

SEQ ID NO: 105 - DNA, 53 bp, synthetic construct
  source 1..53: mol_type = other DNA
tcgtcggcagcgtcagatgtgtataagagacagggcagagcttgctgctctag

SEQ ID NO: 106 - DNA, 54 bp, synthetic construct
  source 1..54: mol_type = other DNA
gtctcgtgggctcggagatgtgtataagagacaggacaagctcaagtccagctg

SEQ ID NO: 107 - DNA, 52 bp, synthetic construct
  source 1..52: mol_type = other DNA
tcgtcggcagcgtcagatgtgtataagagacaggatggtcagttgcatggac

SEQ ID NO: 108 - DNA, 54 bp, synthetic construct
  source 1..54: mol_type = other DNA
gtctcgtgggctcggagatgtgtataagagacagggtgacctgataccatgccg

SEQ ID NO: 109 - DNA, 53 bp, synthetic construct
  source 1..53: mol_type = other DNA
tcgtcggcagcgtcagatgtgtataagagacagggaagtcatcagcaaagcag

SEQ ID NO: 110 - DNA, 54 bp, synthetic construct
  source 1..54: mol_type = other DNA
gtctcgtgggctcggagatgtgtataagagacagccaacattgttacgcattcc

SEQ ID NO: 111 - DNA, 52 bp, synthetic construct
  source 1..52: mol_type = other DNA
tcgtcggcagcgtcagatgtgtataagagacaggggttcctcagaatcccag

SEQ ID NO: 112 - DNA, 54 bp, synthetic construct
  source 1..54: mol_type = other DNA
gtctcgtgggctcggagatgtgtataagagacagcatccattcttcagagcagg

SEQ ID NO: 113 - DNA, 53 bp, synthetic construct
  source 1..53: mol_type = other DNA
tcgtcggcagcgtcagatgtgtataagagacagcggagaacactgatcttccc

SEQ ID NO: 114 - DNA, 53 bp, synthetic construct
  source 1..53: mol_type = other DNA
gtctcgtgggctcggagatgtgtataagagacagcttccagtgtctgacggtg

SEQ ID NO: 115 - DNA, 52 bp, synthetic construct
  source 1..52: mol_type = other DNA
tcgtcggcagcgtcagatgtgtataagagacagcgtttcagggcagagcgtg

SEQ ID NO: 116 - DNA, 54 bp, synthetic construct
  source 1..54: mol_type = other DNA
gtctcgtgggctcggagatgtgtataagagacagcagcaatccaagttcatcag

SEQ ID NO: 117 - DNA, 52 bp, synthetic construct
  source 1..52: mol_type = other DNA
tcgtcggcagcgtcagatgtgtataagagacagcgtcagcaaagcagaaagc

SEQ ID NO: 118 - DNA, 54 bp, synthetic construct
  source 1..54: mol_type = other DNA
gtctcgtgggctcggagatgtgtataagagacagcctggtccaacattgttacg

SEQ ID NO: 119 - DNA, 53 bp, synthetic construct
  source 1..53: mol_type = other DNA
tcgtcggcagcgtcagatgtgtataagagacaggatggatgggaagtgtatgc

SEQ ID NO: 120 - DNA, 54 bp, synthetic construct
  source 1..54: mol_type = other DNA
gtctcgtgggctcggagatgtgtataagagacagcaattaacagaacctccgtc

SEQ ID NO: 121 - DNA, 53 bp, synthetic construct; identical to SEQ ID NO: 113
  source 1..53: mol_type = other DNA

SEQ ID NO: 122 - DNA, 53 bp, synthetic construct
  source 1..53: mol_type = other DNA
gtctcgtgggctcggagatgtgtataagagacagctcttccaatgtctgacgg

SEQ ID NO: 123 - DNA, 51 bp, synthetic construct
  source 1..51: mol_type = other DNA
tcgtcggcagcgtcagatgtgtataagagacagcagggcggagcgtgttgc

SEQ ID NO: 124 - DNA, 54 bp, synthetic construct
  source 1..54: mol_type = other DNA
gtctcgtgggctcggagatgtgtataagagacagcagcaacccaagttcatcag

SEQ ID NO: 125 - DNA, 17 bp, synthetic construct
  source 1..17: mol_type = other DNA
ccaacgaagacctcgag

SEQ ID NO: 126 - DNA, 17 bp, synthetic construct
  source 1..17: mol_type = other DNA
ccaaagaagacctcgag

SEQ ID NO: 127 - DNA, 17 bp, synthetic construct
  source 1..17: mol_type = other DNA
ctcacgcccaactaaac

SEQ ID NO: 128 - DNA, 17 bp, synthetic construct
  source 1..17: mol_type = other DNA
ctcacgcccaacaaaac

SEQ ID NO: 129 - DNA, 17 bp, synthetic construct
  source 1..17: mol_type = other DNA
gtatttgcacccgcaga

SEQ ID NO: 130 - DNA, 17 bp, synthetic construct
  source 1..17: mol_type = other DNA
gtatttgcacctgcaga

SEQ ID NO: 131 - DNA, 16 bp, synthetic construct
  source 1..16: mol_type = other DNA
tccaagaccagagcta

SEQ ID NO: 132 - DNA, 16 bp, synthetic construct
  source 1..16: mol_type = other DNA
tccaagaccggagcta

SEQ ID NO: 133 - DNA, 16 bp, synthetic construct
  source 1..16: mol_type = other DNA
ctcagtgtgtacgctg

SEQ ID NO: 134 - DNA, 16 bp, synthetic construct
  source 1..16: mol_type = other DNA
ctcagtgtgtatgctg

SEQ ID NO: 135 - DNA, 17 bp, synthetic construct
  source 1..17: mol_type = other DNA
caagcaaacatcttgac

SEQ ID NO: 136 - DNA, 17 bp, synthetic construct
  source 1..17: mol_type = other DNA
caagcgaccatcttgac

SEQ ID NO: 137 - DNA, 17 bp, synthetic construct
  source 1..17: mol_type = other DNA
catgccgattgtggaac

SEQ ID NO: 138 - DNA, 17 bp, synthetic construct
  source 1..17: mol_type = other DNA
catgccaattgttgaac

SEQ ID NO: 139 - DNA, 17 bp, synthetic construct
  source 1..17: mol_type = other DNA
ctctttccccatgaagt

SEQ ID NO: 140 - DNA, 17 bp, synthetic construct
  source 1..17: mol_type = other DNA
ctccttccccatgaagt

SEQ ID NO: 141 - DNA, 17 bp, synthetic construct
  source 1..17: mol_type = other DNA
aataggaatgtgctgac

SEQ ID NO: 142 - DNA, 17 bp, synthetic construct
  source 1..17: mol_type = other DNA
aataggaatgtgttgac

SEQ ID NO: 143 - DNA, 17 bp, synthetic construct
  source 1..17: mol_type = other DNA
caaagaacccgtgtctt

SEQ ID NO: 144 - DNA, 17 bp, synthetic construct
  source 1..17: mol_type = other DNA
caaagaacctgtgtctt

SEQ ID NO: 145 - DNA, 16 bp, synthetic construct
  source 1..16: mol_type = other DNA
ctgctctggccgttga

SEQ ID NO: 146 - DNA, 16 bp, synthetic construct
  source 1..16: mol_type = other DNA
ctgctctagccgttga

SEQ ID NO: 147 - DNA, 16 bp, synthetic construct
  source 1..16: mol_type = other DNA
atccaaacagcccatc

SEQ ID NO: 148 - DNA, 16 bp, synthetic construct
  source 1..16: mol_type = other DNA
atccagacagcccatc

SEQ ID NO: 149 - DNA, 17 bp, synthetic construct
  source 1..17: mol_type = other DNA
ctcagcatattatatgc

SEQ ID NO: 150 - DNA, 17 bp, synthetic construct
  source 1..17: mol_type = other DNA
gcatataatacgatgag

SEQ ID NO: 151 - DNA, 17 bp, synthetic construct
  source 1..17: mol_type = other DNA
tctgtggattcgaattt

SEQ ID NO: 152 - DNA, 17 bp, synthetic construct
  source 1..17: mol_type = other DNA
tctggggatttgaattt

SEQ ID NO: 153 - DNA, 17 bp, synthetic construct
  source 1..17: mol_type = other DNA
atgagataagtctaggt

SEQ ID NO: 154 - DNA, 17 bp, synthetic construct
  source 1..17: mol_type = other DNA
atgagattagtctaggt

SEQ ID NO: 155 - DNA, 17 bp, synthetic construct
  source 1..17: mol_type = other DNA
gaggatcaataggtgta

SEQ ID NO: 156 - DNA, 17 bp, synthetic construct
  source 1..17: mol_type = other DNA
gaggatcaaaaggtgta

SEQ ID NO: 157 - DNA, 17 bp, synthetic construct
  source 1..17: mol_type = other DNA
aagtttgagatgaggtc

SEQ ID NO: 158 - DNA, 17 bp, synthetic construct
  source 1..17: mol_type = other DNA
aagttcgagatgaggtc

SEQ ID NO: 159 - DNA, 16 bp, synthetic construct
  source 1..16: mol_type = other DNA
gccagctcgaggtcaa

SEQ ID NO: 160 - DNA, 16 bp, synthetic construct
  source 1..16: mol_type = other DNA
gccagttcgaggtcaa

SEQ ID NO: 161 - DNA, 16 bp, synthetic construct
  source 1..16: mol_type = other DNA
gaaatccagcaaagca

SEQ ID NO: 162 - DNA, 16 bp, synthetic construct
  source 1..16: mol_type = other DNA
gaaatccagcgaagca

SEQ ID NO: 163 - DNA, 17 bp, synthetic construct
  source 1..17: mol_type = other DNA
tggcattctggttgccc

SEQ ID NO: 164 - DNA, 17 bp, synthetic construct
  source 1..17: mol_type = other DNA
gggaaaccagaatacca

SEQ ID NO: 165 - DNA, 17 bp, synthetic construct
  source 1..17: mol_type = other DNA
aacctccgcttcccagt

SEQ ID NO: 166 - DNA, 17 bp, synthetic construct
  source 1..17: mol_type = other DNA
aacctcctcctcccagt

SEQ ID NO: 167 - RNA, 36 nt, synthetic construct
  source 1..36: mol_type = other RNA
gtcggaacgctcaacgattgcccctcacgaggggac

SEQ ID NO: 168 - RNA, 36 nt, synthetic construct
  source 1..36: mol_type = other RNA
ggagagatctcaaacgattgctcgattagtcgagac

SEQ ID NO: 169 - RNA, 36 nt, synthetic construct
  source 1..36: mol_type = other RNA
accaaaacgactattgattgcccagtacgctgggac

(19) United States
(12) Patent Application Publication
(10) Pub. No.: US 2026/0078392 A1
(43) Pub. Date: Mar. 19, 2026

(54) Genome Editing Method for Duplicated Genes

(71) Applicant: NATIONAL INSTITUTE OF ADVANCED INDUSTRIAL SCIENCE AND TECHNOLOGY, Tokyo (JP)

(72) Inventors: Shigeo Sugano, Tsukuba-shi (JP); Reika Hasegawa, Tsukuba-shi (JP)

(73) Assignee: NATIONAL INSTITUTE OF ADVANCED INDUSTRIAL SCIENCE AND TECHNOLOGY, Tokyo (JP)

(21) Appl. No.: 19/109,216

(22) PCT Filed: Sep. 6, 2023
(86) PCT No.: PCT/JP2023/032542

§ 371 Date: Mar. 6, 2025

(30) Foreign Application Priority Data
Sep. 6, 2022 (JP) 2022-141724

(51) Int. Cl. C12N 15/82; C12N 9/22; C12N 15/11

(52) U.S. Cl. CPC C12N 15/8213; C12N 9/226; C12N 15/11; C12N 2310/20

(57) ABSTRACT

The purpose of the present invention is to provide a technology for, by using a small number of gRNA or crRNA, simultaneously editing numerous genes that are functionally duplicated. It was found that a CasΦ protein derived from huge phages can cut a target sequence having a nucleotide length of 16 or more, and a mismatch sequence including a mismatch of 1 or 2 bases with respect to the target sequence. Accordingly, provided is a genome editing method that is for duplicated genes and that uses said CasΦ protein.

### CROSS REFERENCE TO RELATED APPLICATIONS

This application is a national stage application (under 35 U.S.C. § 371) of PCT/JP2023/032542, filed on Sep. 6, 2023, which claims the benefit of Japanese Patent Application No. 2022-141724 filed Sep. 6, 2022, each of which is incorporated herein by reference in its entirety.

### SUBMISSION OF SEQUENCE LISTING

The instant application contains a Sequence Listing XML which has been submitted electronically and is hereby incorporated by reference in its entirety. Sequence Listing XML copy, created on Mar. 24, 2025, is named “01001300002US.xml” and is 164,388 bytes in size.

### FIELD

The present invention relates to a genome editing method for duplicated genes.

### BACKGROUND

Genome editing is a technique for introducing a mutation into a target region of the genome of an organism. This technique is advantageous in terms of efficiency of obtaining desired genotype, compared to existing mutagenesis methods such as radiation, and is therefore used for a wide range of purposes.

In typical genome editing techniques such as CRISPR/Cas9 and CRISPR/Cas12a, the sequence of the editing target is largely determined by the site known as the “spacer sequence” of the gRNA or crRNA. The gRNA or crRNA spacer sequence used is usually a freely modifiable target sequence of 20 to 24 nucleotides. In addition to the spacer sequence, a 2 to 4 nucleotide sequence known as a “PAM sequence” is also necessary for the nuclease Cas9 or Cas12a to function. Genome editing is therefore done by specifically recognizing a nucleotide sequence with a total of 22 to 28 nucleotides including the PAM sequence and spacer sequence. The recognition length is a length sufficient to specify a single region in the human genome.

In most organisms including plants, however, a phenomenon is observed in which homologous genes present in the genome perform compensating functions (functional redundancy), and consequently simple disruption of a single gene often does not result in appearance of the desired phenotype. In order to express the desired phenotype, therefore, it is usually necessary to simultaneously edit multiple homologous genes with sequence similarity.

The method commonly used for simultaneous editing of multiple genes is to express multiple gRNA or crRNA sequences, specifying genes each with the different gRNA or crRNA. In this method, a greater number of genes as the editing target requires expression of larger number of gRNA or crRNA, and therefore more effort for vector construction.

Another method for simultaneous editing of multiple genes is to use the off-target effect of CRISPR. This method makes use of the ability of gRNA or crRNA of CRISPR/Cas9 to cleave mismatch sequences. Specifically, if a gRNA sequence targeting 20 to 24 nucleotides is designed so as to edit multiple different genes each with a 1 to 2 nucleotide mismatch, then it is possible to simultaneously edit multiple genes with a single gRNA or crRNA. Methods utilizing this off-target effect are advantageous compared to methods using a single gRNA or crRNA for a single gene, in that less effort is required for vector construction since multiple genes can be edited with fewer gRNA or crRNA.

However, methods utilizing an off-target effect are also known to have certain constraints. With CRISPR/Cas9, it has been reported that a shorter spacer sequence corresponds to a lower off-target effect. For Streptococcus pyogenes Cas9 (hereunder referred to as SpyCas9), using gRNA or crRNA having a spacer sequence that recognizes 20 nucleotides may cleave sequences with 1 or 2 mismatches, but using gRNA or crRNA having a spacer sequence that recognizes 17 nucleotides cleaves sequences with mismatches, but with drastically reduced the efficiency for genome editing (NPL 1: Nat Biotechnol. 2014 March; 32 (3): 279-284). As of the current time, therefore, it has been necessary to select either a method of simultaneously editing mismatch sequences by recognition of PAM sequence+20 nucleotides, or a method of simultaneously editing homologous genes completely matched by PAM sequence+17 nucleotides.

A novel Cas has been reported, as a small-sized Cas family known as CasΦ, from huge phages, among which CasΦ2 has been reported to cleave short target sequences of 16 residues in in vitro experiments (NPL 2: Science (2020) 369, 333-337, NPL 3: Nature Communications (2021) volume 12, Article number: 4476). In light of further research on CasΦ based on crystal structure analysis, it has been shown that the nuclease activity is increased in vCasΦ lacking the 155-position to 176-position corresponding to the helix which is predicted to interact with the non-target strand (NPL 4: Nature Structural & Molecular Biology (2021) vol. 28, pp. 652-661). Genome editing of plants using CasΦ has also been reported (NPL 5: Int. J. Mol. Sci. (2022), 23, 5755).

### CITATION LIST

### Non Patent Literature

[NPL 1] Nat Biotechnol. 2014 March; 32 (3): 279-284
[NPL 2] Science (2020) 369, 333-337
[NPL 3] Nature Communications (2021) volume 12, Article number: 4476
[NPL 4] Nature Structural & Molecular Biology (2021) vol. 28, pp. 652-661
[NPL 5] Int. J. Mol. Sci. (2022), 23, 5755
[NPL 6] Plant Biotechnology (2016) 33, 235-243

### SUMMARY

### Technical Problem

Techniques for simultaneous editing of multiple genes, and especially techniques taking advantage of off-target effects, are based on the assumption that genes with functional redundancy have a certain degree of mutual sequence similarity. However, it is a known empirical fact that the sequence similarity of the redundant genes is not sufficiently high in actuality.

The present inventors analyzed genomic sequence data for Arabidopsis thaliana, as a particularly representative example of a plant with the problem of functional redundancy, and examined the extent of continuous matching between the sequences of the duplicated genes. As a result, it was found that a near majority of genes are those wherein the length of continuous nucleotide sequence matching is less than 24 nucleotides. In attempting to disrupt a homologous gene group with functional redundancy, a problem was found with CRISPR/Cas9 or CRISPR/Cas12a, in that either the recognition length is too long or the off-target effect is too weak.

It is therefore an object of the present invention to solve the problem described above by providing a technique for simultaneous editing of multiple genes with overlapping function, using small variety of gRNA or crRNA.

### Solution to Problem

As a result of diligent research conducted with the aim of achieving the object stated above, it was found that CasΦ protein derived from huge phage has a nucleotide target length of 16 or greater and is able to cleave a target sequence containing 0 to 2 mismatches within cells, and the invention of a genome editing method for duplicated genes was thereupon devised.

The present invention relates to the following:

[1] A genome editing method for duplicated genes in a cell, wherein the duplicated genes have only a 16 nucleotide length target sequence for genome editing, or a 16 or 17 nucleotide length target sequence and a mismatch sequence containing 1 to 2 nucleotide mismatches with the target sequence, and the method comprises a step of expressing in cells:
(a) crRNA containing a spacer sequence targeting the target sequence and mismatch sequence; and
(b) CasΦ protein derived from a huge phage and able to cleave the target sequence and mismatch sequence.

[2] The genome editing method according to [1] above, wherein the CasΦ protein is at least one protein selected from the group consisting of CasΦ1, CasΦ2, vCasΦ and CasΦ3.

[3] The genome editing method according to [1] above, wherein the CasΦ protein includes an amino acid sequence having at least 80% sequence homology with an amino acid sequence selected from the group consisting of SEQ ID NO: 1, 2, 3 and 4.

[4] The genome editing method according to any one of [1] to [3] above, wherein the duplicated genes are duplicated sequences with functional redundancy.

[5] The genome editing method according to any one of [1] to [4] above, wherein the duplicated genes include a PAM sequence near the target sequence.

[6] The genome editing method according to any one of [1] to [5] above, wherein the spacer sequence is determined so as to completely match the target sequence.

[7] The genome editing method according to any one of [1] to [6] above, wherein the duplicated genes have a target sequence with a nucleotide length of 16 or greater, and optionally a mismatch sequence containing 1 to 2 nucleotide mismatches with the target sequence.

[8] The genome editing method according to any one of [1] to [7] above, wherein the duplicated genes have a target sequence with a nucleotide length of 17 or greater and a mismatch sequence containing 1 to 2 nucleotide mismatches with the target sequence.

[9] The genome editing method according to any one of [1] to [8] above, wherein the duplicated genes are present in 2 to 100 copies in the genome of the cell.

[10] The genome editing method according to any one of [1] to [9] above, wherein all or some of the duplicated genes are edited, among the duplicated genes in the genome of the cell.

[11] The genome editing method according to any one of [1] to above, wherein the cells are plant cells.

[12] The genome editing method according to any one of [1] to above, wherein the crRNA and/or CasΦ protein are expressed by being introduced into the cell using an expression vector.

[13] The genome editing method according to above, wherein the expression vector is a transient or constitutive expression vector.

[14] A kit for genome editing of duplicated genes in a cell, wherein the duplicated genes have only a 16 nucleotide length target sequence for genome editing, or a 16 or 17 nucleotide length target sequence and a mismatch sequence containing 1 to 2 nucleotide mismatches with the target sequence, and the kit comprises:
(a) an expression cassette for crRNA which is able to transfer a spacer sequence targeting the target sequence and mismatch sequence; and
(b) an expression cassette for CasΦ protein derived from a huge phage and able to cleave the target sequence and mismatch sequence.

### Advantageous Effects of Invention

According to the invention it is possible to simultaneously edit multiple homologous genes using a small number of crRNA.

### BRIEF DESCRIPTION OF DRAWINGS

FIG. 1A is a graph showing duplicated genes of Arabidopsis thaliana in groups, classified according to continuous matching nucleotide sequence length. A total of 1383 duplicated gene groups were extracted from the PTGbase. The abscissa represents continuous matching nucleotide sequence length, and the ordinate represents number of groups. FIG. 1B shows a graph for the continuous matching nucleotide sequence lengths on the abscissa of FIG. 1A, magnified between the 1 to 60 residue lengths. The number of duplicated gene groups with continuous matching of less than 30 nucleotides was 818, and when PAM is included, the probability of simultaneously targeting many duplicated genes is low.

FIG. 2A shows a vector map for pEX-35S-HPT.

FIG. 2B shows a vector map for pEX-CasPhi2-HPT.

FIG. 3 is a graph showing editing efficiency with different nucleotide lengths of the spacer sequence, for genome editing using CasΦ2 and vCasΦ.

FIG. 4A shows genome editing analysis results for the target site, obtained by agroinfiltration of tobacco leaves. Specifically, FIG. 4(A) shows mutations introduced with NbPDS-1 as the target.

FIG. 4B shows genome editing analysis results for the target site, obtained by agroinfiltration of tobacco leaves. Specifically, FIG. 4(B) shows mutations introduced with NbPDS-2 as the target.

FIG. 4C shows genome editing analysis results for the target site, obtained by agroinfiltration of tobacco leaves. Specifically, FIG. 4(C) shows mutations introduced with NbRDR6-1 as the target.

FIG. 4D shows genome editing analysis results for the target site, obtained by agroinfiltration of tobacco leaves. Specifically, FIG. 4(D) shows mutations introduced with NbRDR6-2 as the target.

FIG. 4E shows genome editing analysis results for the target site, obtained by agroinfiltration of tobacco leaves. Specifically, FIG. 4(E) shows mutations introduced with NbSGS3-1 as the target.

FIG. 4F shows genome editing analysis results for the target site, obtained by agroinfiltration of tobacco leaves. Specifically, FIG. 4(F) shows mutations introduced with NbSGS3-2 as the target.

FIG. (A) shows the amino acid sequence for CasΦ2 (SEQ ID NO: 1). FIG. 5(B) shows the amino acid sequence for CasΦ1 (SEQ ID NO: 2). FIG. 5(C) shows the amino acid sequence for CasΦ3 (SEQ ID NO: 3).

FIG. 5(D) shows an Arabidopsis thaliana codon-optimized CasΦ2 DNA sequence (uppercase)+DNA sequences coding for the nuclear localization signal and FLAG tag (SEQ ID NO: 9).

FIG. 5(E) shows the nucleotide sequence for the coding region of CasΦ2 on a constructed plasmid (SEQ ID NO: 10).

FIG. 5(F) shows the amino acid sequence for the coding region of CasΦ2 on a constructed plasmid (SEQ ID NO: 5). FIG. 5(G) shows an Arabidopsis thaliana codon-optimized vCasΦ DNA sequence (uppercase)+DNA sequences coding for the nuclear localization signal and FLAG tag (lowercase) (SEQ ID NO: 11).

FIG. 5(H) shows the amino acid sequence for the coding region of vCasΦ on a constructed plasmid (SEQ ID NO: 6). FIG. 5(I) shows the crRNA sequence for CasΦ2 and vCasΦ (SEQ ID NO: 12) (uppercase indicating the direct repeat sequence; spacer sequence added at the 3′-end). FIG. 5(J) shows the crRNA sequence for CasΦ1 (SEQ ID NO: 13) (uppercase indicating the direct repeat sequence; spacer sequence added at the 3′-end). FIG. 5(K) shows the crRNA sequence for CasΦ3 (SEQ ID NO: 14) (uppercase indicating the direct repeat sequence; spacer sequence added at the 3′-end). FIG. 5(L) shows the amino acid sequence for vCasΦ (SEQ ID NO: 4).

### DESCRIPTION OF EMBODIMENTS

One aspect of the invention relates to a genome editing method for duplicated genes. More specifically, the duplicated genes to be edited by the genome editing method for duplicated genes of the invention are further characterized by having a 16 or 17 nucleotide length target sequence as the target of genome editing, and/or a mismatch sequence containing 1 to 2 nucleotide mismatches with the target sequence. The genome editing method for duplicated genes of the invention comprises a step of expressing the following in cells containing duplicated genes:
(a) crRNA containing a spacer sequence targeting the target sequence and mismatch sequence; and
(b) CasΦ protein derived from a huge phage and able to cleave the target sequence and mismatch sequence. The method of the invention allows target sequences of duplicated genes to be recognized and cleaved, by intracellular expression of crRNA and CasΦ, thereby allowing genome editing to be accomplished. The method of the invention is preferably used for cells of an organism having numerous duplicated genes. Such organisms include plants, animals and fungi. Particularly preferred plants include cereals, horticultural crops, ornamental plants and feed plants, and plants that are industrially useful in particular include Poaceae plants such as rice, wheat, barley, wild oat, rye, millet, foxtail millet, barnyard millet, corn and sugarcane, Fabaceae plants such as soybean, Polygonaceae plants such as buckwheat, Cucurbitaceae plants such as cucumber, watermelon, pumpkin and zucchini, Solanaceae plants such as eggplant, tomato, potato, capsicum, bell pepper and tobacco, Convolvulaceae plants such as sweet potato, Araceae plants such as taro and satoimo, Dioscoreaceae plants such as Japanese yam, yam and nagaimo, Brassicaceae plants such as cabbage, Chinese cabbage, turnip, Japanese radish and thale cress (Arabidopsis thaliana), Lamiaceae plants such as perilla, basil and rosemary, Rosaceae plants such as cherry flower, plum, peach, strawberry, apple and pear, Umbelliferae plants such as ginseng, celery, cumin, mitsuba and parsley, Amaryllidaceae plants such as onion, Welsh onion and garlic, and Euphorbiaceae plants such as cassava.

According to one aspect, the duplicated genes are two or more homologous genes present in a single genome. Homologous genes are genes deriving from the same ancestral gene and having the similar structure or function. According to one aspect, duplicated genes may be defined as genes having nucleotide sequences with at least 60%, preferably at least 70% and more preferably at least 80% sequence homology (preferably sequence identity). According to another aspect, duplicated genes may be defined as genes either containing common nucleotide sequences of at least a specified length, and/or containing nucleotide sequences with 1 or 2 nucleotide mismatches with respect to a nucleotide sequence of at least a specified length. The “specified length” may be selected as appropriate, an example being 16 or more nucleotides, 17 or more nucleotides or 18 or more nucleotides. The duplicated genes are preferably genes with functional redundancy. A group of multiple homologous genes is referred to as a duplicated gene group. Duplicated genes are frequently present as repeats on a single chromosome, but they may also be present on different chromosomes by translocation. A duplicated gene group for genome editing has multiple genes sharing a target sequence and/or a mismatch sequence containing 1 to 2 nucleotide mismatches with the target sequence. From the viewpoint of accomplishing genome editing for all duplicated gene groups that are to be gene edited, with a single genome editing operation, it is preferred for all of the genes in the duplicated gene group to share the target sequence and/or mismatch sequences. The number of duplicated genes in the duplicated gene group for genome editing is not particularly restricted, but it will usually be 2 to 100 copies. The number of duplicated genes in the duplicated gene group for genome editing may be, for duplicated genes arising from whole genome duplication, 3 or more copies or 5 or more copies, and 50 or fewer copies or 20 or fewer copies, and preferably 10 or fewer copies. By sharing the target sequence, the genome editing method of the invention allows genome editing by cleavage of all or a portion of the target sequence and/or mismatch sequences of multiple genes in a duplicated gene group. The genes in the duplicated gene group for genome editing preferably have high sequence similarity between them.

According to the invention, a target sequence is sequence corresponding to the crRNA spacer sequence in the genome editing method of the invention, and it is associated with the sequence which is to be cleaved by the genome editing method of the invention for genome editing. The target sequence is a common sequence among the duplicated genes in the duplicated gene group, and may comprise a continuous sequence with a length of 16 or 17 nucleotides. The target sequence is a sequence that completely matches among the duplicated gene group. A mismatch sequence containing 1 or 2 mismatches with respect to the target sequence may also be cleaved for genome editing by the genome editing method of the invention. The target sequence must be set so that the spacer sequence of crRNA is near the target sequence, particularly with the PAM sequence being included adjacent to the 5′-end. The PAM sequence may be a PAM sequence that recognizes CasΦ, such as TTN or NTN, for example.

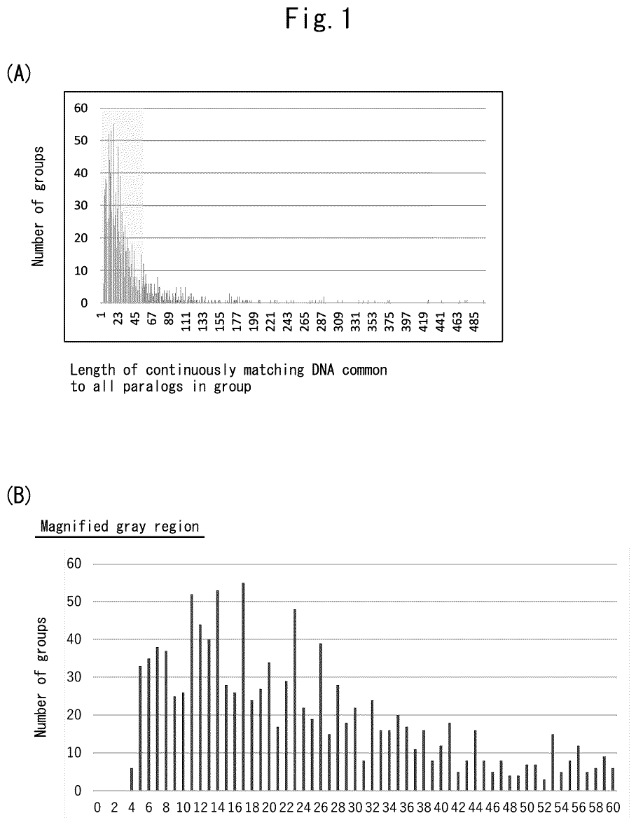

In the genome editing method, the target sequence and mismatch sequence are cleaved by a single crRNA spacer sequence, allowing genome editing. Genome editing of all or part of the target sequence and mismatch sequence in the duplicated gene group is possible. Arabidopsis thaliana has 1383 duplicated gene groups, among which 818 duplicate gene groups have continuous matching of less than 30 nucleotides (FIG. 1A). When using conventional Cas9, therefore, it is difficult to accomplish genome editing with a single crRNA spacer sequence when dealing with many duplicated genes. On the other hand, approximately 70% of the duplicated gene groups in Arabidopsis thaliana have a common sequence with a continuous nucleotide length of 16 or greater. If 1 or 2 mismatches are allowed, then genome editing can be accomplished by a single crRNA spacer sequence in more than about 80% of duplicated gene groups. Also, about 65% of the duplicated gene groups in Arabidopsis thaliana have a common sequence with a continuous nucleotide length of 17 or greater. If 1 or 2 mismatches are allowed, then genome editing can be accomplished by a single crRNA spacer sequence in more than about 75% of duplicated gene groups. The nucleotide length of the target sequence may vary depending on the CasΦ used.

When the target sequence has a sequence with a nucleotide length of 16, the target sequence may be set so that the duplicated gene group for genome editing consists of genes containing only the target sequence, or the target sequence may be set so that it further includes genes containing mismatch sequences.

When the target sequence has a sequence with a nucleotide length of 17, on the other hand, the target sequence may be set so that the duplicated gene group for genome editing includes genes containing the target sequence and genes containing the mismatch sequence.

The crRNA referred to here is RNA containing a spacer sequence comprising the same nucleotide sequence as the target sequence. The crRNA forms a complex with Cas protein, and is guided to the target sequence or mismatch sequence of genomic DNA, with the activity of Cas protein enabling cleavage of DNA between PAM and the target sequence or mismatch sequence. The crRNA sequence used may be selected according to the type of Cas protein used. While any publicly known crRNA sequence for CasΦ protein may be used, an example is a sequence selected from among sequences consisting of SEQ ID NO: 12, SEQ ID NO: 13 and SEQ ID NO: 14 (the RNA sequences for which are SEQ ID NO: 167, SEQ ID NO: 168 and SEQ ID NO: 169), depending on the type of CasΦ protein. As an example, the sequence of SEQ ID NO: 12 may be used for CasΦ2 and vCas. When CasΦ1 is used, the sequence of SEQ ID NO: 13 may be used. When CasΦ3 is used, the sequence of SEQ ID NO: 14 may be used. The crRNA can be transiently or constitutively expressed using the expression cassette described below.

CasΦ protein is a protein of the huge phage-derived Cas family. CasΦ protein has activity of cleaving target sequences with a nucleotide length of 16 or greater, and also mismatch sequences containing 1 to 2 nucleotide mismatches with target sequences. The upper limit for the length of a target sequence to be cleaved by CasΦ protein is not particularly restricted, but from the viewpoint of cleavage activity it will usually be 20 or less, preferably 18 or less and more preferably 17 or less. CasΦ can be selected from the group consisting of CasΦ1, CasΦ2 and CasΦ3, but CasΦ2 (also known as Cas12j2) is most preferably used from the viewpoint of off-target activity. The CasΦ may also have an amino acid sequence mutation (such as a substitution, deletion or addition) so long as the activity is not completely impaired. From this viewpoint, the CasΦ protein may have the amino acid sequence selected from the group consisting of SEQ ID NO: 1, SEQ ID NO: 2, SEQ ID NO: 3 and SEQ ID NO: 4, or a sequence with at least 60%, at least 70%, at least 80%, at least 90%, at least 93%, at least 95%, at least 97%, at least 98% or at least 99% sequence homology (preferably sequence identity) with the sequence. For CasΦ2 in particular, mutant CasΦ2 with a deletion of the helix region (positions 155-176 of SEQ ID NO: 1) which is thought to interact with the non-target strand (also known as vCasΦ: amino acid sequence: SEQ ID NO: 4) is more preferred for increased cleavage reaction speed (NPL 3).

As used herein, “homology” between two amino acid sequences is the percentage of equal or similar amino acid residues appearing at corresponding sites when the two amino acid sequences have been aligned, while “identity” between two amino acid sequences is the percentage of equal amino acid residues appearing at corresponding sites when the two amino acid sequences have been aligned. The “homology” and “identity” of two amino acid sequences can be determined using BLAST (Basic Local Alignment Search Tool) program (Altschul et al., J. Mol. Biol., (1990), 215 (3): 403-10).

A CasΦ protein having a sequence identified by sequence homology or identity is further specified by desired cleavage activity. The cleavage activity can be specified as the cleavage activity of a target sequence with a nucleotide length of 16 or greater and a mismatch sequence containing 1 to 2 nucleotide mismatches with the target sequence, either in vitro or in vivo. The cleavage activity of the CasΦ protein can be evaluated by a publicly known in vitro method. Since the RMSD value between the CasΦ2 backbone and the CasΦ3 backbone, when using PDB_ID: 7odf (CasΦ3) and 7lys (CasΦ2), is less than 1.135 A based on calculation by PyMOL software alignment, and 0.97 when further including nucleic acid, the structures of CasΦ2 and CasΦ3 are highly similar. Since the TM-score was also ≥0.85 with independent software, this supports the conclusion that the structures are similar. Variants can therefore be identified by the RMSD value in addition to, or instead of, identification based on sequence identity or homology. When the Cα mean square deviation (RMSD) between the backbone of one or more Cas proteins selected from the group consisting of CasΦ1, CasΦ2, CasΦ3 and vCasΦ and the backbone of a variant is less than 2.5 Å, preferably less than 1.5 Å and more preferably less than 1 Å, there is a high probability that the effect exhibited will be the same as that of the original Cas protein. Identification by TM-score value is also possible. When the TM-score between the backbone of one or more Cas proteins selected from the group consisting of CasΦ1, CasΦ2, CasΦ3 and vCasΦ and the backbone of a variant is 0.8 or greater, preferably 0.85 or greater and more preferably 0.9 or greater, there is a high probability that the effect exhibited will be the same as that of the original Cas protein.

Additional sequences such as a signal sequence, tag sequence and reporter sequence may also be included, so long as the desired activity of the CasΦ protein is not impaired. From the viewpoint of ensuring that the expressed protein functions in the nucleus, preferably a nuclear localization signal is added as a signal sequence. A nuclear localization signal may have one or more nuclear localization signals arranged in series. The nuclear localization signal may be selected as appropriate for the plant variety, and examples are KRPAATKKAGQAKKKK (SEQ ID NO: 7) and/or GSDYKDHDGDYKDHDIDYKDDDDKPKKKRKV (SEQ ID NO: 8). The CasΦ protein expressed in cells can migrate into the nucleus, cooperating with crRNA in the nucleus to cleave the target sequence.

The crRNA or expression cassette will generally be prepared so as to include a promoter sequence, with a crRNA-coding sequence placed under its control. The crRNA expression cassette may be present on a single polynucleotide, or it may be present on different polynucleotides. The crRNA may be a crRNA expression cassette having a sequence coding for the crRNA and multiple linked single-stranded guide RNAs, placed under the control of a promoter sequence.

The CasΦ protein may also be expressed in the cells either transiently or constitutively, according to an established method. As an example, a polynucleotide with a sequence coding for the CasΦ protein may be set in an expression cassette containing a promoter sequence that allows transient or constitutive expression, under the control of a promoter, to prepare a CasΦ expression cassette. Placement under the control of a promoter means placement so that expression is controlled by the promoter, and it may be placement at a suitable number of nucleotides, such as 10 bp to 200 bp, from the 3′-end of the promoter.

The crRNA expression cassette and CasΦ expression cassette may be present on a single polynucleotide, or they may be present on different polynucleotides. The expression cassettes may also contain other elements as necessary. Such elements include elements necessary for expression, such as a terminator, as well as elements necessary for preparation of a plasmid or vector, such as a multicloning site, drug resistance gene, reporter gene and replication origin.

The promoter used in the crRNA expression cassette and CasΦ expression cassette may be selected as appropriate for the expressing species. For expression in a plant, a polII promoter may be used, for example, but a polIII promoter is preferred from the viewpoint of more accurately carrying out transcription of relatively short RNA. Examples of polII promoters include CaMV35S promoter, RPS5A promoter, UBQ promoter, DD45 promoter and NOS promoter. Examples of polIII promoters include U6-snRNA (such as U6.1-snRNA and U6.26-snRNA) promoter, and U3-snRNA promoter.

The terminator used for the crRNA expression cassette and CasΦ expression cassette is not particularly restricted so long as it is a nucleotide sequence that is able to terminate transcription from the Cas protein coding sequence (preferably a polyA sequence added to mRNA transcribed from the Cas protein coding sequence). The guide RNA may have a polyT sequence added. The termination signal may be, for example, heat shock protein termination signal (HspT: Heat shock protein Terminator), NosT (Noparin synthase Terminator), 35sT (CaMV 35S Terminator) or Pea3A (Pea Rubisco subunit 3A).

Another aspect of the invention relates to a method for producing a crRNA expression cassette comprising a step of determining a target sequence for a duplicated gene group for genome editing. The target sequence for the duplicated gene group may be selected so that the continuous sequence of 16 nucleotides near the PAM sequence of CasΦ contains no mismatches, or it may be selected so as to allow 1 or 2 mismatches, among the duplicated gene group for genome editing, based on genomic information. According to another aspect, the target sequence for the duplicated gene group may be selected so that the continuous sequence of 17 nucleotides near the PAM sequence of CasΦ is composed of a sequence without mismatches, and a mismatch sequence that contains 1 or 2 mismatches, among the duplicated gene group for genome editing, based on genomic information. The crRNA sequence may be designed by using the determined target sequence as the spacer sequence and linking it with the remaining sequences. A polynucleotide comprising the designed crRNA sequence can be easily created by a publicly known genetic engineering method. A polynucleotide comprising the created crRNA sequence or guide RNA sequence can be incorporated into a crRNA expression cassette using a publicly known genetic engineering method such as PCR, restriction enzyme treatment, DNA linkage or in vitro transcription.

Another aspect of the invention relates to a kit for genome editing of duplicated genes in a cell. In the kit, the duplicated gene for genome editing either has only a 16 nucleotide length target sequence, or both a 16 or 17 nucleotide length target sequence and a mismatch sequence containing 1 to 2 nucleotide mismatches with the target sequence. The kit includes the following:
(a) an expression cassette for crRNA into which a spacer sequence targeting the target sequence and mismatch sequence can be introduced; and
(b) an expression cassette for Cas protein derived from a huge phage and able to cleave the target sequence and mismatch sequence. The crRNA expression cassette and CasΦ protein expression cassette are incorporated into a plasmid or vector, for example, and gene transferred into cells. The kit for genome editing according to the invention may further include other materials, reagents, or instruments necessary for the genome editing method of the invention, such as a nucleic acid delivery reagent or buffer, as appropriate and necessary. Other materials necessary for carrying out the genome editing method of the invention include a donor polynucleotide, in addition to the crRNA expression cassette and/or CasΦ protein expression cassette. By introducing a donor polynucleotide into the nucleus, it is possible to knock-in the donor polynucleotide at a cleavage site produced by the CRISPR/Cas system.

Another aspect of the invention relates to a genome editing method for duplicated genes in a cell, the method comprising:
(a) a step of delivering into the cell a crRNA expression cassette produced by the method for producing a crRNA expression cassette, and
(b) a step of delivering into the cell a CasΦ expression cassette coding for CasΦ protein that is able to cleave an expression cassette for CasΦ protein derived from a huge phage and able to cleave the target sequence and mismatch sequence. The crRNA expression cassette and CasΦ expression cassette may be separately or simultaneously transferred into two polynucleotides, or they may be simultaneously transferred into a single polynucleotide. A donor polynucleotide may also be delivered, in addition to delivering the crRNA expression cassette and/or CasΦ protein expression cassette into cells. By introducing a donor polynucleotide into the nucleus, it is possible to knock-in the donor polynucleotide at a cleavage site produced by the CRISPR/Cas system.

The gene delivery can be carried out by a publicly known method in the genome engineering technical field. The delivery method is not particularly restricted and may be appropriately selected according to the type of entity to delivered and the target of deliver. Delivery methods are largely classified as direct methods or viral vector methods. A direct method may be a PEG method or electroporation method utilizing the phagocytosis effect of protoplasts lacking the cell walls of plant cells, or a particle gun method in which injection is done with gold particles, or the whisker method. Methods using viral vectors include the use of vectors such as Agrobacterium, tobacco mosaic virus (TMV), plum pox virus (PPV), potato X virus (PVX), alfalfa mosaic virus (AIMV), cucumber mosaic virus (CMV), cowpea mosaic virus (CPMV), zucchini yellow mosaic virus (ZYMV) and gemini virus, depending on the host plant variety. An Agrobacterium method is preferred among these from the viewpoint of convenience and safety. In addition to vectors such as those mentioned above which have been adapted for delivery into plant bodies or plant cells, other examples include vectors designed for delivery of the polynucleotide of the invention into such vectors (for example, Gateway® entry clone vector).

The cells used as the delivery target may be cells cultured in vitro, or cells forming the plant body. The site of delivery in the plant body may be the flowers (especially flower egg cells or pollen), leaves, roots, seeds or embryo. The shoot apex is preferable as the delivery target from the viewpoint of efficient genome editing in germ cells, so that mutations produced by genome editing are propagated to the next generation. It is also preferred to use a method of redifferentiation from the gene delivered cells to obtain genome-edited individuals, or a method of activating genes that induce shoot apexes from somatic cells to obtain genome-edited individuals.

All of the publications mentioned throughout the present specification are incorporated herein in their entirety by reference.

The present invention will now be explained in further detail by Examples, with the understanding that these are merely provided for convenience of explanation and are not intended to limit the invention in any sense.

### EXAMPLES

### Example 1: Preparation of Vectors for Huge Phage-Derived CasΦ2 and vCasΦ

### (1) Preparation of Huge Phage-Derived CasΦ2 Gene

A DNA sequence coding for Cas protein with codon optimization for Arabidopsis thaliana, with addition of a DNA sequence coding for the C-terminal nuclear localization signal sequence-FLAG tag (SEQ ID NO: 9), was artificially synthesized based on amino acid sequence information derived from a huge phage (SEQ ID NO: 1). The Sanger method or a next-generation sequencing method was used to determine the nucleotide sequence. The amino acid sequence listed as SEQ ID NO: 1 can be found in a well-known database such as GenBank of the NCBI (National Center for Biotechnology Information).

### (2) Construction of Transient CasΦ2 Expression Vector for Plant

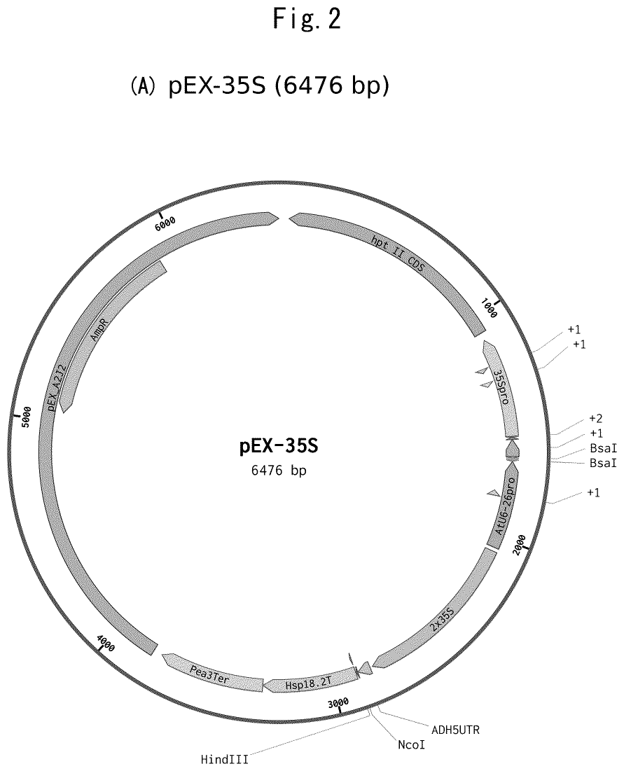

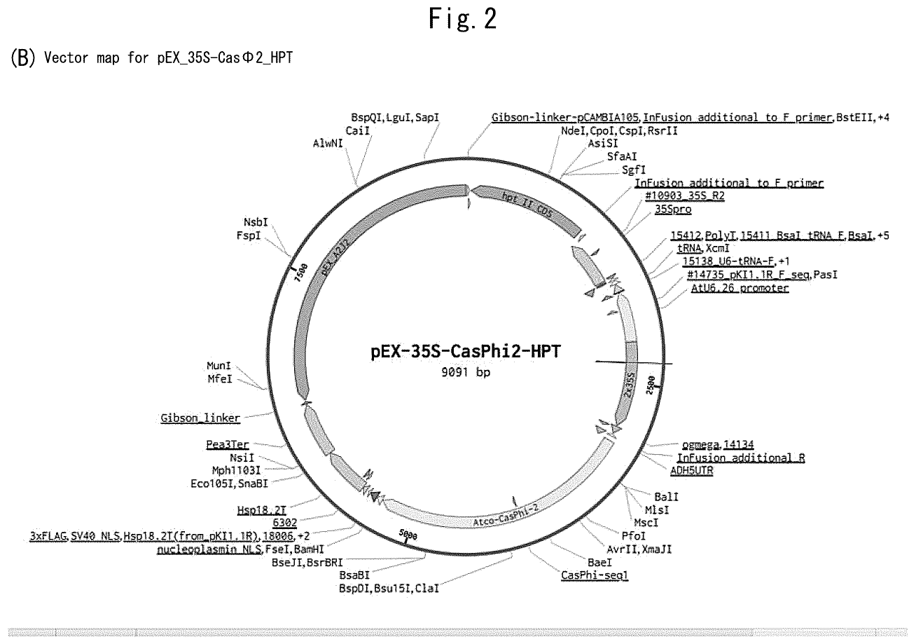

The Arabidopsis thaliana codon-optimized Cas gene (SEQ ID NO: 9) having the nuclear localization signal-FLAG tag added was incorporated into pEX_35S_vector (gene synthesis by Eurofins Genomics KK.: FIG. 2A). The synthetic gene fragment of SEQ ID NO: 9 and the linearized pEX_35S_vector cut with NcoI and HindIII were mixed, and the DNA fragments were formed into a circular plasmid using NEBuilder HiFi DNA Assembly Master Mix. E. coli DH5a was transformed with the obtained circular plasmid and cultured on an LB agar plate containing 50 μg/mL ampicillin to select the transformants. Several colonies were picked up and clones possessing the target circular plasmid were selected, purifying the plasmids. Below are shown the nucleotide sequence of the CasΦ coding sequence of the constructed vector (SEQ ID NO: 10), and the corresponding amino acid sequence (SEQ ID NO: 5). The vectors were then cut with BsaI and NruI to obtain linear DNA. At the same time, double-stranded DNA was prepared by annealing oligonucleotides having the following sequences:

TABLE 1

| | | |
|---|---|---|
| 18133 | CGAGAGTGTCGTTCGtaaaaaaaagagaccctctctcggtctccGTCCCCTCGTGAGGG | (Seq ID No. 15) |
| 18134 | CCCTCACGAGGGGAGggagaccgagagagggtctcttttttttaCGAACGACACTCTCG | (Seq ID No. 16) |

For each type, the double-stranded DNA annealed with the cut vector was formed into a circular plasmid using T4 DNA ligase. The circular plasmid was purified by this procedure. This vector was designated as pEX_35S-CasΦ2_HPT (FIG. 2B).
(3) Construction of Transient vCasΦ Expression Vector for Plant

The Arabidopsis thaliana codon-optimized CasΦ gene (SEQ ID NO: 9) with the nuclear localization signal-FLAG tag added was divided by PCR.

Primer Sets with the Following Sequences:

TABLE 2

| | |
|---|---|
| Set1-forward | TCCTCCAGAGACTTTCCGCTTCTTCTTTGGGGCTTTATGATTCATATGTATATCTCCTTGTTAAAG |
| | (Seq ID No. 17) |
| Set1-Reverse | TCTGGCGGCTCAAAAAGAACCGCCGACGGCAGCGAATTCGAGCCCAAGAAGAAGAGGAAAGTCCTCGAGCACCACCAG |
| | (Seq ID No. 18) |
| Set2-forward | TCCTCCAGAGACTTTCCGCTTCTTCTTTGGGGCTTTATGATTCATATGTATATCTCCTTCTTAAAG |
| | (Seg ID No. 19) |
| Set2-Reverse | TCTGGCGGCTCAAAAAGAACCGCCGACGGCAGCGAATTCGAGCCCAAGAAGAAGAGGAAAGTCCTCGAGCACCACCAC |
| | (Seq ID No. 20) |

were used for amplification by PCR reaction using the synthetic gene fragment of SEQ ID NO: 9 as template. Two linear DNAs that could be amplified by PCR reaction were mixed with linearized pEX_Cas9 vector cut with NcoI and HindIII, and the DNA fragments were formed into a circular plasmid using NEBuilder HiFi DNA Assembly Master Mix. E. coli DH5a was transformed with the obtained circular plasmid and cultured on an LB agar plate containing 50 μg/mL ampicillin to select the transformants. Several colonies were picked up and clones possessing the target circular plasmid were selected, purifying the plasmids. Below are shown both the nucleotide sequence of the vCasΦ coding sequence of the constructed vector (SEQ ID NO: 11) and the corresponding amino acid sequence (SEQ ID NO: 6). This vector was designated as pEX_35S-vCasΦ_HPT.
(4) CasΦ2 and vCasΦ Vector Assemblies for Plant Individuals

pCAMBIA105.1R (Cosmo Bio Co., Ltd.) was cut with PmlI and AseI to obtain a linear binary vector. At the same time, pEX_35S-CasΦ2_HPT and pEX_35S-vCasΦ_HPT were each cut with PaqCl to obtain linear DNA. Each DNA was mixed with the linear binary vector, and the DNA fragments were formed into a circular plasmid using NEBuilder HiFi DNA Assembly Master Mix. E. coli DH5a was transformed with the obtained circular plasmid and cultured on an LB agar plate containing 50 μg/mL ampicillin to select the transformants. Several colonies were picked up and clones possessing the target circular plasmid were selected, purifying the plasmids. This vectors were designated as pCA_35S-CasΦ2_HPT and pCA_35S-vCasΦ_HPT.

### Example 2: Mutagenesis in Arabidopsis thaliana Protoplast Cells Using CasΦ2 and vCasΦ

(1) Preparation of Plasmids for Genome Editing Experiment of Arabidopsis thaliana Gene

The pEX_35S-CasΦ2_HPT vector or pEX_35S-vCasΦ_HPT vector was cut with BsaI-HF. First, in order to examine whether genome editing was possible with these vectors, a total of three different double-stranded DNAs were prepared, being annealed to different sets of oligonucleotides having the sequences listed in Table 3:

TABLE 3

| | | |
|---|---|---|
| Set1-Phi-PDS3-33_F | ggacCAGTTGACAATCCAGCCA | (Seq ID No. 21) |
| Set1-Phi-PDS3-33_R | acaaTGGCTGGATTGTCAACTG | (Seq ID No. 22) |
| Set2-Phi-PDS3-33-16n-F | ggacCAGTTGACAATCCAGC | (Seq ID No. 23) |
| Set2-Phi-PDS3-33-16n-R | acaaGCTGGATTGTCAACTG | (Seq ID No. 24) |
| Set3-Phi-PDS3-33-15n-F | ggacCAGTTGACAATCCAG | (Seq ID No. 25) |
| Set3-Phi-PDS3-33-15n-R | acaaCTGGATTGTCAACTG | (Seq ID No. 26) |

for cloning of the spacer sequence of CasΦ crRNA for a genome editing experiment using the Arabidopsis thaliana-derived PDS3 gene (a gene with a single copy in the Arabidopsis thaliana genome). The PDS3 gene is the phytoene desaturase 3 gene, a carotenoid biosynthase, whose knockout is known to result in whitening of plant cells. This allows the location of the knockout in plant bodies to be identified. For each type, the double-stranded DNA annealed with the cut vector was formed into a circular plasmid using T4 DNA ligase. E. coli DH5a was transformed with the obtained circular plasmid and cultured on an LB agar plate containing 50 μg/mL ampicillin to select transformants. Several colonies were picked up and clones possessing the target circular plasmid were selected, purifying the plasmids.
(2) Preparation of Arabidopsis thaliana Protoplasts

Arabidopsis thaliana leaves were grown for about 20 to 30 days, and after stripping the epidermis with cellophane tape to expose the mesophyll cells, a suitable amount of the leaves was immersed in a solution containing 1.0% Cellulase Onozuka R10 (Yakult Honsha Co., Ltd.), 0.25% Macerozyme R-10 (Yakult Honsha Co., Ltd.), 10 mM mercaptoethanol, 400 mM mannitol, 20 mM KCl, 10 mM CaCl₂) and 20 mM MES (pH 5.7), and shake cultured at 22° C., 50 rpm while incubating for 1 hour, to free the protoplasts. The protoplasts were filtered with a 70 μm-pore size nylon filter and then collected in a 50 ml-volume tube, and after recovering the protoplasts by centrifugation at 100×g for 10 minutes and discarding the supernatant, they were resuspended in buffer (W5 buffer) containing 150 mM NaCl, 125 mM CaCl₂, 5 mM KCl and 2 mM MES (pH 5.7). The resuspended protoplasts were overlaid in an 18% sucrose solution and centrifuged at 400×g for 5 minutes to concentrate only the healthy cells. The concentrated cells were resuspended in W5 buffer. After twice washing with W5 buffer, the cells were incubated at 4° C. for 10 minutes. The incubated protoplasts were centrifuged at 100×g for 5 minutes and then resuspended in buffer containing 400 mM mannitol, 15 mM MgCl₂ and 4 mM MES (pH 5.7). After then recovering the protoplasts by centrifugation at 100×g for 5 minutes, the cell concentration was adjusted to 2.0 to 3.0×10⁵ cells/ml with buffer to obtain a protoplast suspension for transformation.

(3) Delivery of Plasmid DNA into Arabidopsis thaliana Protoplasts

After mixing 35 μL of the obtained protoplast suspension for transformation with 10 μL of a 10 μM plasmid solution, 45 μL of a solution containing 40% (w/v) PEG4000, 200 mM mannitol and 100 mM CaCl₂ was placed in each well of a 96-well plate (round-bottom, product of Nunc Co.), and shaking was carried out at 900 rpm for 15 seconds to mix the solution. The liquid mixture was allowed to stand at room temperature for 10 minutes. After suspending the protoplasts by vigorously adding 200 μL of W5 buffer to the standing liquid mixture, they were centrifuged at 100×g for 5 minutes, and the protoplast cells were collected at the plate bottom, discarding 200 μL of the supernatant and washing the protoplast suspension. After repeating this procedure a total of 4 times, each well was sealed with Parafilm and allowed to stand at 22° C. for 36 hours.

### (4) Amplicon-Seq Analysis of Genomic DNA

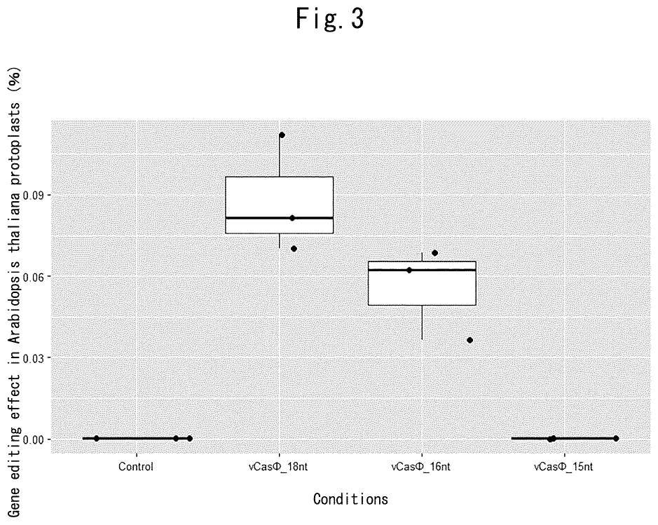

After delivery of the plasmid DNA, the protoplast suspension that had been allowed to stand at 22° C. for 36 hours was collected in a 200 μL-volume tube and mixed with genome extraction buffer (200 mM Tris-HCl, pH 7.5, 250 mM NaCl, 25 mM EDTA, 0.5% SDS), after which it was incubated at 95° C. for 10 minutes and then allowed to stand for 5 minutes on ice. The genomic DNA was recovered by isopropanol precipitation from the heat-treated protoplast suspension. The recovered genomic DNA was suspended in sterilized water and an approximately 300 bp DNA fragment amplified using primer sets having the sequences listed in Table 4 below was treated by Amplicon-seq using an iSeq100 system (Illumina), for detection of genome editing. The editing efficiency with vCasΦ using target sequence lengths changed to 15, 16 and 18 were analyzed with CRISPResso2 software, and the results are shown in FIG. 3. A group with an expression vector for the fluorescent protein GFP was used as the Control. As a further control for vCasΦ, editing efficiency was examined when using SpyCas9 for a target sequence length of 18. While gene editing was possible with a target sequence length of 16 and 18 when using the vCasΦ of FIG. 3, genome editing did not occur with a target sequence length of 15. The genome editing efficiency when using SpyCas9 was about 0.02, but when using vCasΦ it was a maximum of about 0.09.

TABLE 4
Casφ2 or vCasφ, PDS3 Target

| | |
|---|---|
| TCGTCGGCAGCGTCAGATGTGTATAAGAGACAGggaaaagaatcatatggtcatcaattcg | (Seq ID No. 27) |
| GTCTCGTGGGCTCGGAGATGTGTATAAGAGACAGcggacttgaattaaaggggctatagtag | (Seq ID No. 28) |

SpyCas9.PDS3

[symbol]

| | |
|---|---|
| TCGTCGGCAGCGTCAGATGTGTATAAGAGACAGGCAAATAATTAAAGTTGTTGCTGTTGG | (Seq ID No. 29) |
| GTCTCGTGGGCTCGGAGATGTGTATAAGAGACAGtagcctacttgcctgcttttc | (Seq ID No. 30) |

### Example 3: Mutagenesis of Tobacco Leaf Cells with vCasΦ

### (1) Preparation of Plasmids for Genome Editing Experiment of Tobacco Gene.

Vector pEX_35S-vCasΦ_HPT was cut with BsaI-HF. In order to clone a spacer sequence for formation of CasΦ crRNA for a genome editing experiment with tobacco-derived PDS, RDR6 and SGS3 genes, a total of 21 types of double-stranded DNA were prepared, each annealed to different sets of oligonucleotides having the sequences listed in Table 5:

TABLE 5

| | |
|---|---|
| (NbPDS_Set1, 17bases) | |
| 5′-GGACCCAACGAAGACCTCGAG-3′ | (Seq ID No. 31) |
| 5′-ACAACTCGAGGTCTTCGTTGG-3′ | (Seq ID No. 32) |
| (NbPDS_Set2, 17bases) | |
| 5′-GGACCTCACGCCCAACTAAAC-3′ | (Seq ID No. 33) |
| 5′-ACAAGTTTAGTTGGGCGTGAG-3′ | (Seq ID No. 34) |
| (NbPDS_Set3, 17bases) | |
| 5′-GGACGTATTTGCACCCGCAGA-3′ | (Seq ID No. 35) |
| 5′-ACAATCTGCGGGTGCAAATAC-3′ | (Seq ID No. 36) |
| (NbPDS_Set4, 16bases) | |
| 5′-GGACTCCAAGACCAGAGCTA-3′ | (Seq ID No. 37) |
| 5′-ACAATAGCTCTGGTCTTGGA-3′ | (Seq ID No. 38) |
| (NbPDS_Set5, 16bases) | |
| 5′-GGACCCTCAGTGTGTACGCTG-3′ | (Seq ID No. 39) |
| 5′-ACAACAGCGTACACACTGAG-3′ | (Seq ID No. 40) |
| (NbPDS_Set6, 17bases) | |
| 5′-GGACCAAGCAAACATCTTGAC-3′ | (Seq ID No. 41) |
| 5′-ACAAGTCAAGATGTTTGCTTG-3′ | (Seq ID No. 42) |
| (NbPDS_Set7, 17bases) | |
| 5′-GGACCATGCCGATTGTGGAAC-3′ | (Seq ID No. 43) |
| 5′-ACAAGTTCCACAATCGGCATG-3′ | (Seq ID No. 44) |
| (NbRDR6_Set1, 17bases) | |
| 5′-GGACCTCTTTCCCCATGAAGT-3′ | (Seq ID No. 45) |
| 5′-ACAAACTTCATGGGGAAAGAG-3′ | (Seq ID No. 46) |
| (NbRDR6_Set2, 17bases) | |
| 5′-GGACAATAGGAATGTGCTGAC-3′ | (Seq ID No. 47) |
| 5′-ACAAGTCAGCACATTCCTATT-3′ | (Seq ID No. 48) |
| (NbRDR6_Set3, 17bases) | |
| 5′-GGACCAAAGAACCCGTGTCTT-3′ | (Seq ID No. 49) |
| 5′-ACAAAAGACACGGGTTCTTTG-3′ | (Seq ID No. 50) |
| (NbRDR6_Set4, 16bases) | |
| 5′-GGACCTGCTCTGGCCGTTGA-3′ | (Seq ID No. 51) |
| 5′-ACAATCAACGGCCAGAGCAG-3′ | (Seq ID No. 52) |
| (NbRDR6_Set5, 16bases) | |
| 5′-GGACATCCAAACAGCCCATC-3′ | (Seq ID No. 53) |
| 5′-ACAAGATGGGCTGTTTGGAT-3′ | (Seq ID No. 54) |
| (NbRDR6_Set6, 17bases) | |
| 5′-GGACCTCAGCATATTATATGC-3′ | (Seq ID No. 55) |
| 5′-ACAAGCATATAATATGCTGAG-3′ | (Seq ID No. 56) |
| (NbRDR6_Set7, 17bases) | |
| 5′-GGACTCTGTGGATTCGAATTT-3′ | (Seq ID No. 57) |
| 5′-ACAAAAATTCGAATCCACAGA-3′ | (Seq ID No. 58) |
| (NbSGS3_Set1, 17bases) | |
| 5′-GGACATGAGATAAGTCTAGGT-3′ | (Seq ID No. 59) |
| 5′-ACAAACCTAGACTTATCTCAT-3′ | (Seq ID No. 60) |
| (NbSGS3_Set2, 17bases) | |
| 5′-GGACGAGGATCAATAGGTGTA-3′ | (Seq ID No. 61) |
| 5′-ACAATACACCTATTGATCCTC-3′ | (Seq ID No. 62) |
| (NbSGS3_Set3, 17bases) | |
| 5′-GGACAAGTTTGAGATGAGGTC-3′ | (Seq ID No. 63) |
| 5′-ACAAGACCTCATCTCAAACTT-3′ | (Seq ID No. 64) |
| (NbSGS3_Set4, 16bases) | |
| 5′-GGACGCCAGCTCGAGGTCAA-3′ | (Seq ID No. 65) |
| 5′-ACAATTGACCTCGAGCTGGC-3 | (Seq ID No. 66) |
| (NbSGS3_Set5, 16bases) | |
| 5′-GGACGAAATCCAGCAAAGCA-3′ | (Seq ID No. 67) |
| 5′-ACAATGCTTTGCTGGATTTC-3′ | (Seq ID No. 68) |
| (NbSGS3_Set6, 17bases) | |
| 5′-GGACTGGCATTCTGGTTGCCC-3′ | (Seq ID No. 69) |
| 5′-ACAAGGGCAACCAGAATGCCA-3′ | (Seq ID No. 70) |
| (NbSGS3_Set7, 17bases) | |
| 5′-GGACAACCTCCGCTTCCCAGT-3′ | (Seq ID No. 71) |
| 5′-ACAAACTGGGAAGCGGAGGTT-3′ | (Seq ID No. 72) |

The PDS gene is the phytoene desaturase gene, a carotenoid biosynthase, whose knockout is known to result in whitening of plant cells. This allows the location of the knockout in plant bodies to be identified. Two duplicates of the PDS gene are known to exist in tobacco. The RDR6 gene is RNA-dependent RNA polymerase 6 which is involved in distinguishing between exogenous RNA and normal mRNA, and its knockout is known to have an effect of reducing resistance against viruses. Two duplicates of the RDR6 gene are known to exist in tobacco. The SGS3 gene is gene silencing suppressor 3, and it functions in cooperation with RDR6 for protection against viruses. Knockout of the SGS3 gene is known to have an effect of reducing resistance against viruses. Two duplicates of the SGS3 gene are known to exist in tobacco. For each type, the double-stranded DNA annealed with the cut vector was formed into a circular plasmid using T4 DNA ligase. E. coli DH5a was transformed with the obtained circular plasmid and cultured on an LB agar plate containing 50 μg/mL spectinomycin to select the transformants. Several colonies were picked up and clones possessing the target circular plasmid were selected, purifying the plasmids. The purified plasmids were transferred into Agrobacterium GV3101 by electroporation and cultured on an LB agar plate containing 50 μg/mL spectinomycin, 50 μg/mL gentamycin and 50 μg/mL rifampicillin, to select out the transformants.

### (2) Agroinfiltration Experiment in Tobacco Leaves

Colonies of Agrobacterium GV3101 (see Materials and Methods in NPL 6: Plant Biotechnology 33, 235-243 (2016)) having the target circular plasmid were inoculated into liquid LB medium containing 50 μg/mL spectinomycin, 50 μg/mL gentamycin and 50 μg/mL rifampicillin, and shake cultured for 2 days at 28° C. GV3101 having a pDEST_35S_RUBY_HSP (Ab28) construct which expresses red color after gene delivery was cultured. Using the pDEST_35S_RUBY_HSP (Ab28) construct causes red coloration of the regions of the plant body modified by agroinfiltration.

The cultured cells were centrifuged at 3000 rpm for 20 min and collected, and then suspended in about 5 ml of infiltration buffer (composition: 10 mM MgCl₂, 10 mM MES, 100 μM acetosyringone, pH 5.7) and adjusted to about OD600=1. An agroinfiltration solution was prepared by mixing 2 ml of Agrobacteria solution containing the target genome editing vector, 0.5 ml of Ab28-containing Agrobacteria solution, and 2 ml of infiltration buffer.

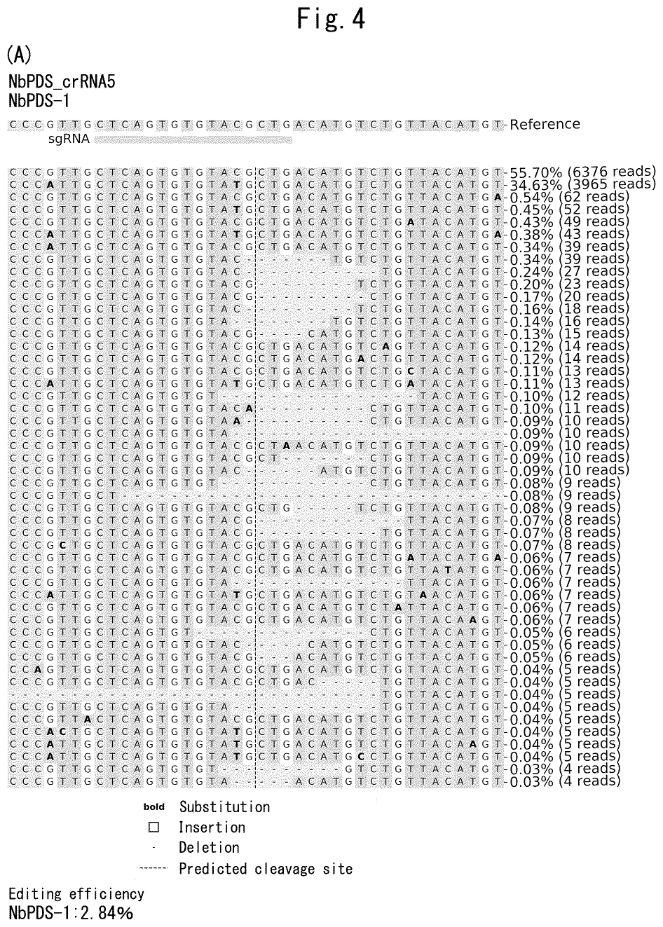

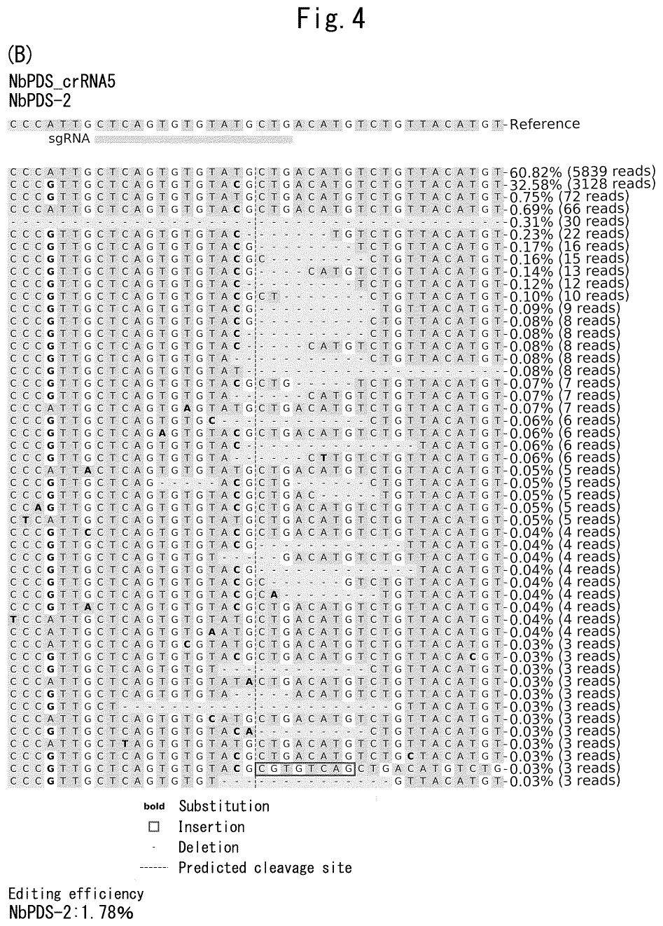

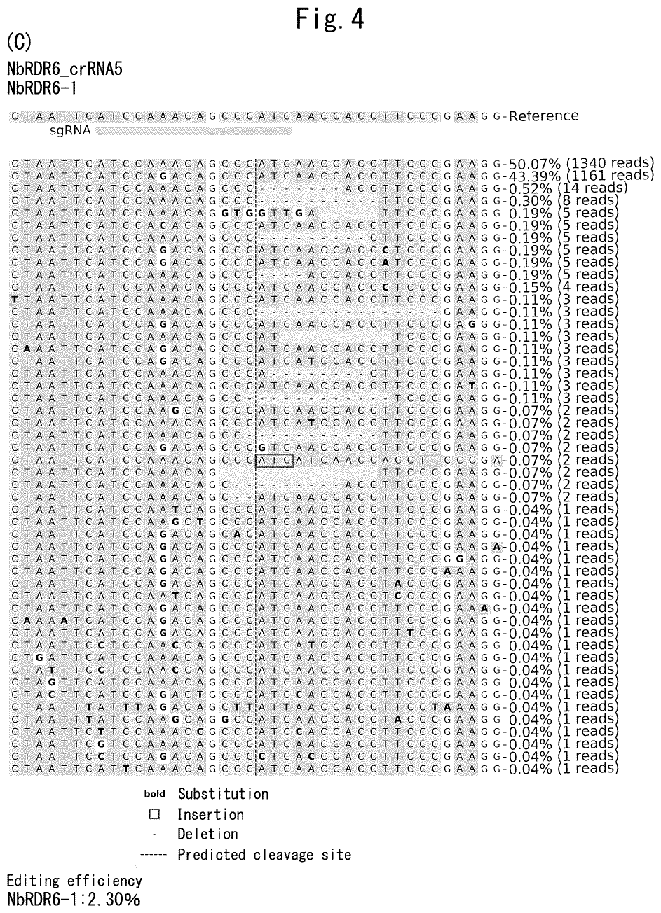

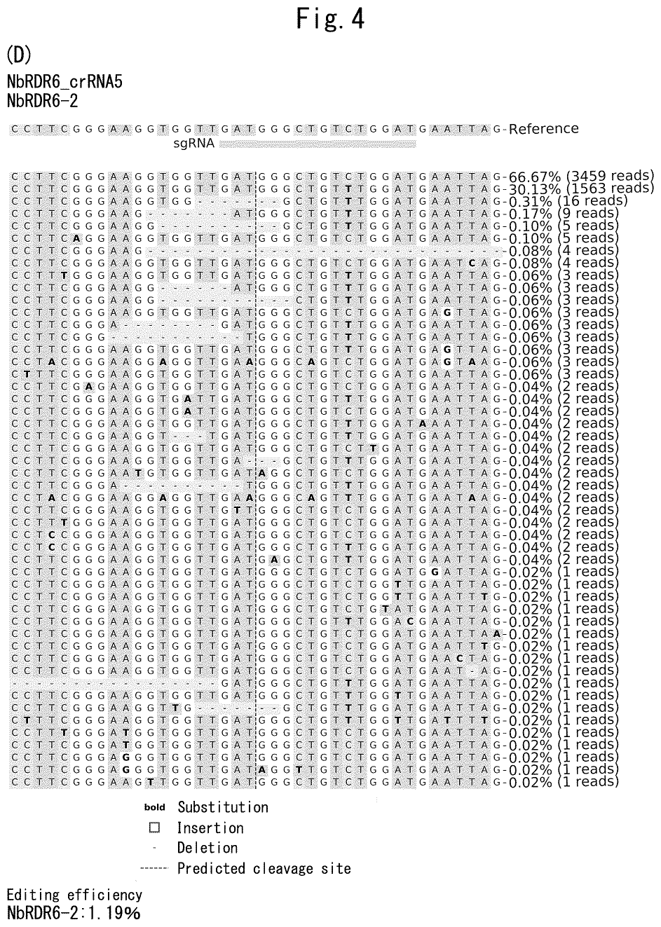

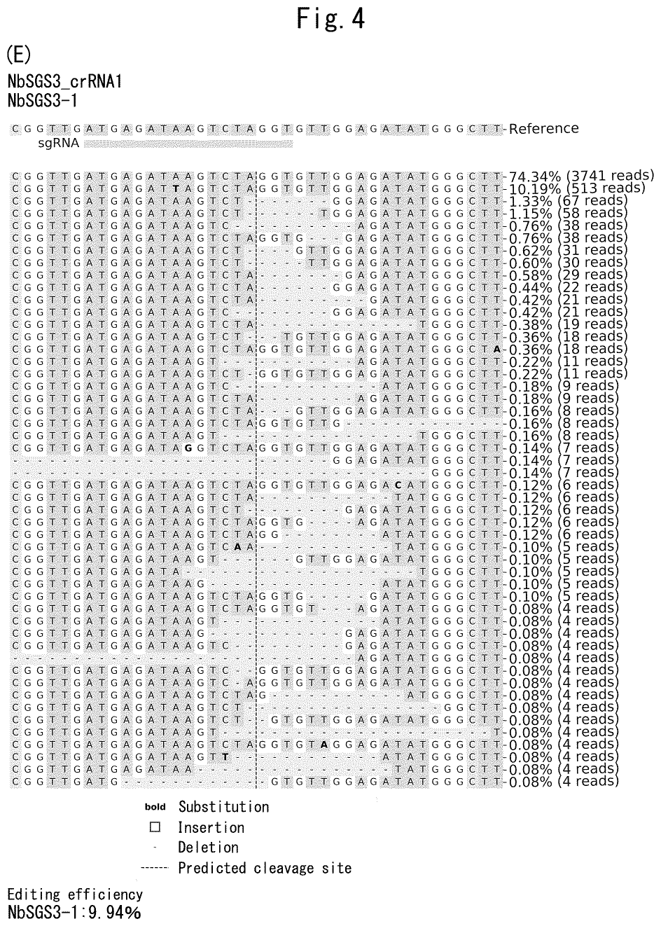

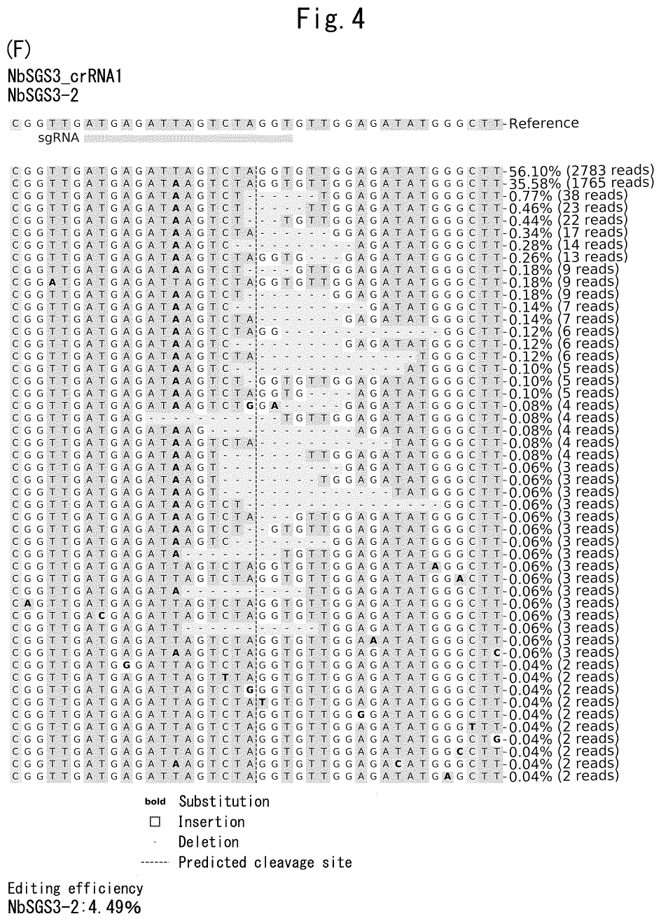

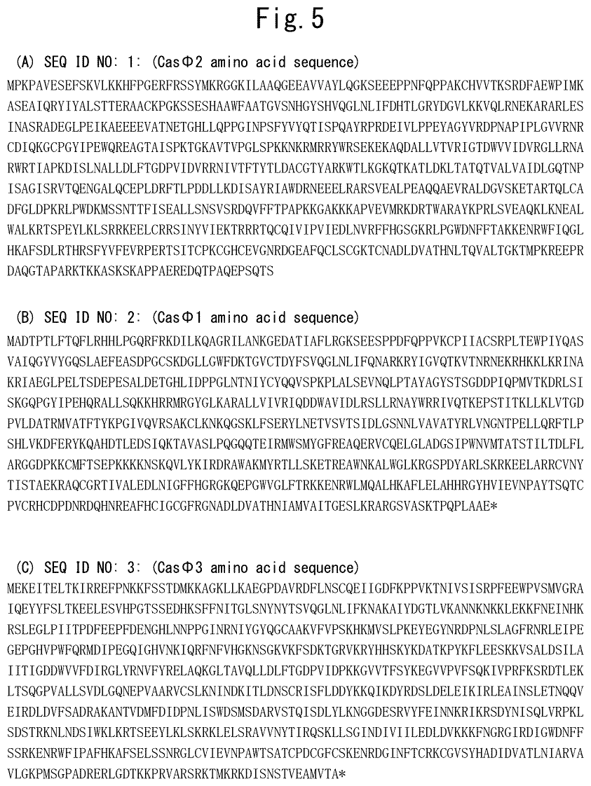

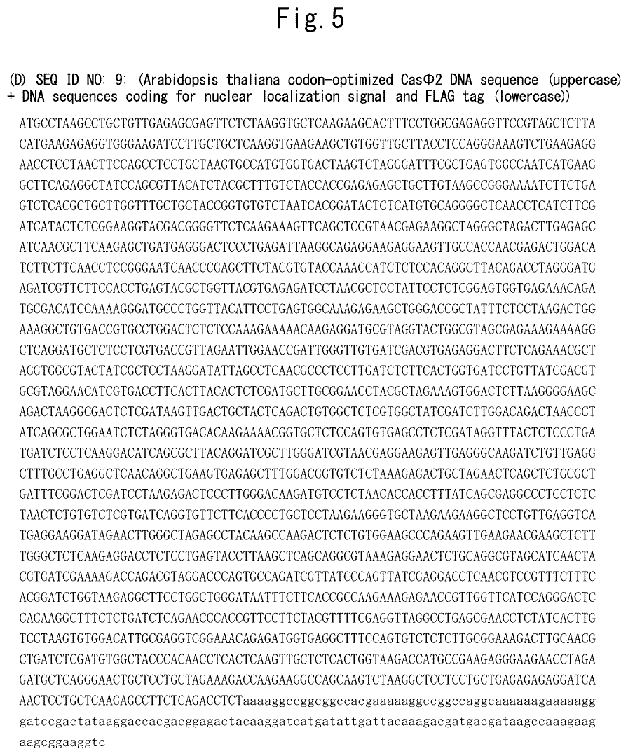

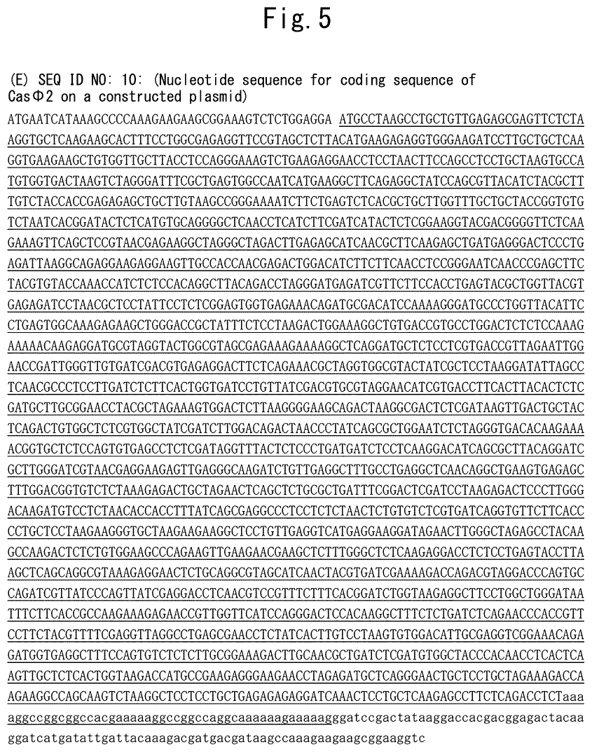

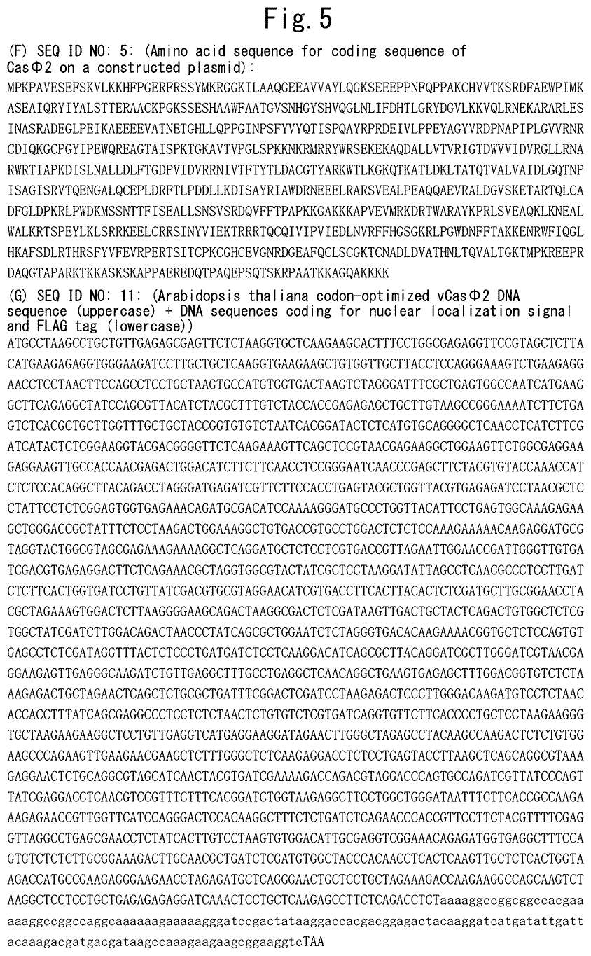

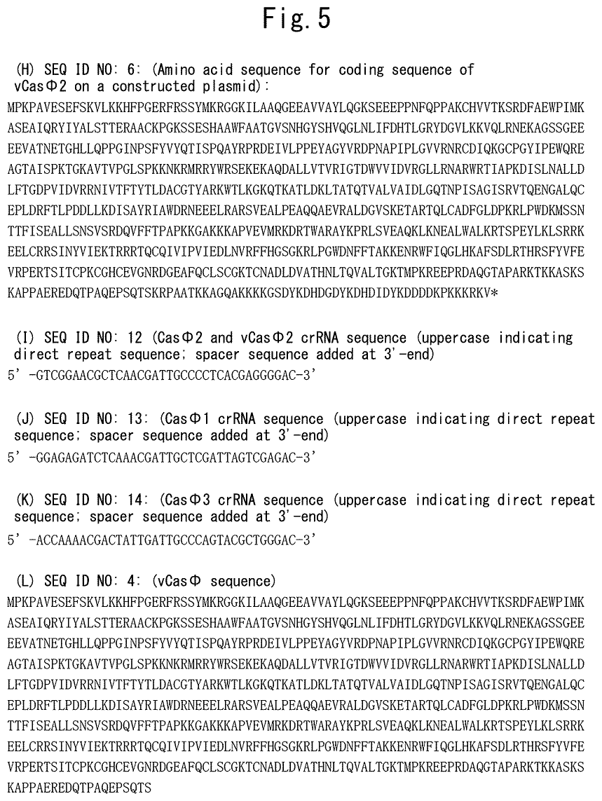

After preparing tobacco plant bodies at 3 weeks after seeding by being grown at 27° C. under long day conditions, the agroinfiltration solution was aspirated with a 1 ml syringe and injected into the back sides of the leaves, incubating for about 3 days at 27° C. under long day conditions, to express the object plasmids (partial results shown in FIG. 4).

### (3) Amplicon-Seq Analysis of Genomic DNA

After then cutting a section of about 1 cm-square from a region where red coloring was exhibited and collecting it in a 2 ml-volume tube, and then mixing with genome extraction buffer (200 mM Tris-HCl, pH 7.5, 250 mM NaCl, 25 mM EDTA, 0.5% SDS) and crushing the tissue slices, they were incubated at 95° C. for 5 minutes and the genomic DNA was recovered by isopropanol precipitation. The recovered genomic DNA was suspended in sterilized water and an approximately 300 bp DNA fragment amplified using primer sets having the sequences listed in Table 6 below was treated with Amplicon-seq using an iSeq100 system (Illumina), for detection of genome editing.

TABLE 6

| | |
|---|---|
| (NbPDS-1_set1) | |
| 5′-tcgtcggcagcgtcagatgtgtataagagacagGCCCCAAATTGGACTTGTTTCTG-3′ | (Seq ID No. 73) |
| 5′-gtctcgtgggctcggagatgtgtataagagacagCCAGCATCACACTTTCGCATTC-3′ | (Seq ID No. 74) |
| (NbPDS-1_set2) | |
| 5′-tcgtcggcagcgtcagatgtgtataagagacagGTGTGATGCTGGATTTATGATCG-3′ | (Seq ID No. 75) |
| 5′-gtctcgtgggctcggagatgtgtataagagacagCTAATAGAATGATCTTCCTTCC-3′ | (Seq ID No. 76) |
| (NbPDS-1_set3) | |
| 5′-tcgtcggcagcgtcagatgtgtataagagacagGGGACCTGAGATATCGGTGC-3′ | (Seq ID No. 77) |
| 5′-gtctcgtgggctcggagatgtgtataagagacagGACACTTCCATCCTCATTCAGC-3′ | (Seq ID No. 78) |
| (NbPDS-1_set4) | |
| 5′-tcgtcggcagcgtcagatgtgtataagagacagGCTACATGTCGATTATGTTCCCC-3′ | (Seq ID No. 79) |
| 5′-gtctcgtgggctcggagatgtgtataagagacagGCAGCAACTCACACACAAATCC-3′ | (Seq ID No. 80) |
| (NbPDS-1_set5) | |
| 5′-tcgtcggcagcgtcagatgtgtataagagacagGGGTCCTCTGATATAACTGGTC-3′ | (Seq ID No. 81) |
| 5′-gtctcgtgggctcggagatgtgtataagagacagCTCGCATACTACADAACTATG-3′ | (Seq ID No. 82) |
| (NbPDS-2_set1) | |
| 5′-tcgtcggcagcgtcagatgtgtataagagacagGCCCCAAATTGGACTTGTTTC-3′ | (Seq ID No. 83) |
| 5′-gtctcgtgggctcggagatgtgtataagagacagGCTCGTGATCATAAATTCAGC-3′ | (Seq ID No. 84) |
| (NbPDS-2_set2) | |
| 5′-tcgtcggcagcgtcagatgtgtataagagacagTGATCACGAGCATATATTCTC-3′ | (Seq ID No. 85) |
| 5′-gtctcglgggctcggagatgtgtataagagacagCTAATAGAATGATCTTCCTTCC-3′ | (Seq ID No. 86) |
| (NbPDS-2_set3) | |
| 5′-tcgtcggcagcgtcagatgtgtataagagacagGACCTGACATATTGGTGCAGG-3′ | (Seq ID No. 87) |
| 5′-gtctcgtgggctcggagatgtgtataagagacagGACACTTCCATCCTCATTCAG-3′ | (Seq ID No. 88) |
| (NbPDS-2_set4) | |
| 5′-tcgtcggcagcgtcagatgtgtataagagacagGGCACCATTGTTGGTGGAGAAAG-3′ | (Seq ID No. 89) |
| 5′-gtctcgtgggctcggagatgtgtataagagacagGCAGGACACAGATCTAGCAGC-3′ | (Seq ID No. 90) |
| (NbPDS-2_set5) | |
| 5′-tcgtcggcagcgtcagatgtgtataagagacagGCCCACTATATCCGTTCATG-3′ | (Seq ID No. 91) |
| 5′-gtctcgtgggctcggagatgtgtataagagacagGCAAATGATTACTGGCCTTGG-3′ | (Seq ID No. 92) |
| (NbRDR6-1_set1) | |
| 5′-tcgtcggcagcgtcagatgtgtataagagacagGTACTTGAGACAGCATCTGG-3′ | (Seq ID No. 93) |
| 5′-gtctcgtgggctcggagatgtgtataagagacagGGACATATATGGTCCATGCC-3′ | (Seq ID No. 94) |
| (NbRDR6-1_set2) | |
| 5′-tcgtcggcagcgtcagatgtgtataagagacagGACAAGCTCAAGTCCAGCAG-3′ | (Seq ID No. 95) |
| 5′-gtctcgtgggctcggagatgtgtataagagacagGTGGTTCATGCTGACCTCAG-3′ | (Seq ID No. 96) |
| (NbRDR6-1_set3) | |
| 5′-tcgtcggcagcgtcagatgtgtataagagacagCATCTACAGCCTCCAGAATC-3′ | (Seq ID No. 97) |
| 5′-gtctcgtgggctcggagctgtgtataagagacagGAGCTTCTCAGCTTGGGGAC-3′ | (Seq ID No. 98) |
| (NbRDR6-1_set4) | |
| 5′-tcgtcggcagcgtcagatgtgtataagagacagGCTGGTTAGCTGAGAATGCC-3′ | (Seq ID No. 99) |
| 5′-gtctcgtgggctcggagatgtgtataagagacagCTGTCTTCCACCGACTGTGG-3′ | (Seq ID No. 100) |
| (NbRDR6-2_set1) | |
| 5′-tcgtcggcagcgtcagatgtgtataagagacagCTTCCACCGACTGTGGAGC-3′ | (Seq ID No. 101) |
| 5′-gtctcgtgggctcggagatgtgtataagagacagGCTGGTTAGCTGAGAATGCC-3′ | (Seq ID No. 102) |
| (NbRDR6-2_set2) | |
| 5′-tcgtcggcagcgtcagatgtgtataagagacagGACCTCAGGAATAAGGCAAG-3′ | (Seq ID No. 103) |
| 5′-gtctcgtgggctcggagatgtgtataagagacagGGCGTAAACCAGGAACATC-3′ | (Seq ID No. 104) |
| (NbRDR6-2_set3) | |
| 5′-tcgtcggcagcgtcagatgtgtataagcgacagGGCAGAGCTTGCTGCTCTAG-3′ | (Seq ID No. 105) |
| 5′-gtctcgtgggctcggagatgtgtataagagacagGACAAGCTCAAGTCCAGCTG-3′ | (Seq ID No. 106) |
| (NbRDR6-2_set4) | |
| 5′-tcgtcggcagcgtcagatgtgtataagagacagGATGGTCAGTTGCATGGAC-3′ | (Seq ID No. 107) |
| 5′-gtctcgtgggctcggagatgtgtataagagacagGGTGACCTGATACCATGCCG-3′ | (Seq ID No. 108) |
| (NbSGS3-1_set1) | |
| 5′-tcgtcggcagcgtcagatgtgtataagagacagGGAAGTCATCAGCAAAGCAG-3′ | (Seq ID No. 109) |
| 5′-gtctcgtgggctcggagatgtgtataagagacagCCAACATTGTTACGCATTCC-3′ | (Seq ID No. 110) |
| (NbSGS3-1_set2) | |
| 5′-tcgtcggcagcgtcagatgtgtataagagacagGGGTTCCTCAGAATCCCAG-3′ | (Seq ID No. 111) |
| 5′-gtctcgtgggctcggagatgtgtataagagacagCATCCATTCTTCAGAGCAGG-3′ | (Seq ID No. 112) |
| (NbSGS3-1_set3) | |
| 5′-tcgtcggcagcgtcagatgtgtataagagacagCGGAGAACACTGATCTTCCC-3′ | (Seq ID No. 113) |
| 5′-gtctcgtgggctcggagatgtgtataagagacagCTTCCAGTGTCTGAGGGTG-3′ | (Seq ID No. 114) |
| (NbSGS3-1_set4) | |
| 5′-tcgtcggcagcgtcagatgtgtataagagacagCGTTTCAGGGCAGAGCGTG-3′ | (Seq ID No. 115) |
| 5′-gtctcgtgggctcggagatgtgtataagagacagCAGCAATCCAAGTTCATCAG-3′ | (Seq ID No. 116) |
| (NbSGS3-2_set1) | |
| 5′-tcgtcggcagcgtcagatgtgtataagagacagCGTCAGCAAAGCAGAAAGC-3′ | (Seq ID No. 117) |
| 5′-gtctcgtgggctcggagatgtgtataagagacagCCTGGTCCAACATTGTTACG-3′ | (Seq ID No. 118) |
| (NbSGS3-2_set2) | |
| 5′-tcgtcggcagcgtcagatgtgtataagagacagGATGGATGGGAAGTGTATGC-3′ | (Seq ID No. 119) |
| 5′-gtctcgtgggctcggagatgtgtataagagacagCAATTAACAGAACCTCCGTC-3′ | (Seq ID No. 120) |
| (NbSGS3-2_set3) | |
| 5′-tcgtcggcagcgtcagatgtgtataagagacagCGGAGAACACTGATCTTCCC-3′ | (Seq ID No. 121) |
| 5′-gtctcgtgggctcggagatgtgtataagagacagCTCTTCCAATGTCTGACGG-3′ | (Seq ID No. 122) |
| (NbSGS3-2_set4) | |
| 5′-tcgtcggcagcgtcagatgtgtataagagacagCAGGGGGGAGCGTGTTGC-3′ | (Seq ID No. 123) |
| 5′-gtctcgtgggctcggagatgtgtataagagacagCAGCAACCCAAGTTCATCAG-3′ | (Seq ID No. 124) |

The results of analysis with CRISPResso2 software are shown in FIG. 4. The results summarized according to each target are shown in Table 7.

TABLE 7
Editing efficiency for each tobacco or RNA

| | | | | | | efficiency |
|---|---|---|---|---|---|---|
| | | | | Length | | Editing |
| crRNA Type | Target gene | Target site (5′-3) | SEQ ID No. | (nt) | Mismatches | (%) |
| NbPDS-1_crRNA1 | NbPDS-1 | CCAACGAAGACCTCGAG | (Seq ID No. 125) | 17 | 0 | 0.04 |
| | NbPDS-2 | CCAAAGAAGACCTCGAG | (Seq ID No. 126) | 17 | 1 | 0.01 |
| NbPDS-1_crRNA2 | NbPDS-1 | CTCACGCCCAACTAAAC | (Seq ID No. 127) | 17 | 0 | 1.02 |
| | NbPDS-2 | CTCACGCCCAACAAAAC | (Seq ID No. 128) | 17 | 1 | 0.01 |
| NbPDS-1_crRNA3 | NbPDS-1 | GTATTTGCACCCGCAGA | (Seq ID No. 129) | 17 | 0 | 1.34 |
| | NbPDS-2 | GTATTTGCACCTGCAGA | (Seq ID No. 130) | 17 | 1 | |
| NbPDS-1_crRNA4 | NbPDS-1 | TCCAAGACCAGAGCTA | (Seq ID No. 131) | 16 | 0 | 0.08 |
| | NbPDS-2 | TCCAAGACCGGAGCTA | (Seq ID No. 132) | 16 | 1 | 0.00 |
| NbPDS-1_crRNA5 | NbPDS-1 | CTCAGTGTGTACGCTG | (Seq ID No. 133) | 16 | 0 | 2.84 |
| | NbPDS-2 | CTCAGTGTGTATGCTG | (Seq ID No. 134) | 16 | 1 | 1.78 |
| NbPDS-1_crRNA6 | NbPDS-1 | CAAGCAAACATCTTGAC | (Seq ID No. 135) | 17 | 0 | 0.19 |
| | NbPDS-2 | CAAGCGACCATCTTGAC | (Seq ID No. 136) | 17 | 2 | 0.01 |
| NbPDS-1_crRNA7 | NbPDS-1 | CATGCCGATTGTGGAAC | (Seq ID No. 137) | 17 | 0 | 0.97 |
| | NbPDS-2 | CATGCGAATTGTTGAAC | (Seq ID No. 138) | 17 | 2 | 0.86 |
| NbRDR6-1_crRNA1 | NbRDR6-1 | CTCTTTCCCGATGAAGT | (Seq ID No. 139) | 17 | 0 | 0.51 |
| | NbRDR6-2 | CTCCTTCCCCATGAAGT | (Seq ID No. 140) | 17 | 1 | 0.04 |
| NbRDR6-1_crRNA2 | NbRDR6-1 | AATAGGAATGTGCTGAC | (Seq ID No. 141) | 17 | 0 | 1.03 |
| | NbRDR6-2 | AATAGGAATGTGTTGAC | (Seq ID No. 142) | 17 | 1 | 0.96 |
| NbRDR6-1_crRNA3 | NbRDR6-1 | CAAAGAACCCGTGTCTT | (Seq ID No. 143) | 17 | 0 | 0.19 |
| | NbRDR6-2 | CAAAGAACCTGTGTCTT | (Seq ID No. 144) | 17 | 1 | 0.05 |
| NbRDR6-1_crRNA4 | NbRDR6-1 | CTGCTCTGGCCGTTGA | (Seq ID No. 145) | 16 | 0 | 0.26 |
| | NbRDR6-2 | CTGCTCTAGCCGTTGA | (Seq ID No. 146) | 16 | 1 | |
| NbRDR6-1_crRNA5 | NbRDR6-1 | ATCCAAACAGCCCATC | (Seq ID No. 147) | 16 | 0 | 2.30 |
| | NbRDR6-2 | ATCCAGACAGCCCATC | (Seq ID No. 148) | 16 | 1 | 3.19 |
| NbRDR6-1_crRNA6 | NbRDR6-1 | CTCAGCATATTATATGC | (Seq ID No. 149) | 17 | 0 | 0.36 |
| | NbRDR6-2 | GCATATAATACGATGAG | (Seq ID No. 150) | 17 | 2 | 0.12 |
| NbRDR6-1_crRNA7 | NbRDR6-1 | TCTGTGGATTCGAATTT | (Seq ID No. 151) | 17 | 0 | 0.04 |
| | NbRDR6-2 | TCTGGGGATTTGAATTT | (Seq ID No. 152) | 17 | 2 | 0.00 |
| NbSGS3-1_crRNA1 | NbSGS3-1 | ATGAGATAAGTCTAGGT | (Seq ID No. 153) | 17 | 0 | 9.94 |
| | NbSGS3-2 | ATGAGATTAGTCTAGGT | (Seq ID No. 154) | 17 | 1 | 4.49 |
| NbSGS3-1_crRNA2 | NbSGS3-1 | GAGGATCAATAGGTGTA | (Seq ID No. 155) | 17 | 0 | 0.10 |
| | NbSGS3-2 | GAGGATCAAAAGGTGTA | (Seq ID No. 156) | 17 | 1 | 0.02 |
| NbSGS3-1_crRNA3 | NbSGS3-1 | AAGTTTGAGATGAGGTC | (Seq ID No. 157) | 17 | 0 | 5.29 |
| | NbSGS3-2 | AAGTTCGAGATGAGGTC | (Seg ID No. 158) | 17 | 1 | 0.50 |
| NbSGS3-1_crRNA4 | NbSGS3-1 | GCCAGCTCGAGGTCAA | (Seq ID No. 159) | 16 | 0 | 0.02 |
| | NbSGS3-2 | GCCAGTTCGAGGTCAA | (Seq ID No. 160) | 16 | 1 | 0.01 |
| NbSGS3-1_crRNA5 | NbSGS3-1 | GAAATCCAGCAAAGCA | (Seq ID No. 161) | 16 | 0 | 0.09 |
| | NbSGS3-2 | GAAATCCAGCGAAGCA | (Seq ID No. 162) | 16 | 1 | 0.01 |
| NbSGS3-1_crRNA6 | NbSGS3-1 | TGGCATTGTGGTTGCCC | (Seq ID No. 163) | 17 | 0 | 0.02 |
| | NbSGS3-2 | GGGAAACCAGAATACCA | (Seq ID No. 164) | 17 | 2 | 0.01 |
| NbSGS3-1_crRNA7 | NbSGS3-1 | AACCTCCGCTTCCCAGT | (Seq ID No. 165) | 17 | 0 | 9.06 |
| | NbSGS3-2 | AACCTCCTCCTCCCAGT | (Seq ID No. 166) | 17 | 2 | 3.39 |

Each target gene was edited with a target length of 16 or 17, and editing was found to have occurred even with sequences containing mismatches.

### [Sequence Listing]

1. A genome editing method for duplicated genes in a cell, wherein the duplicated genes have only a 16-nucleotide length target sequence for genome editing, or a 16- or 17-nucleotide length target sequence for genome editing and a mismatch sequence containing 1 to 2 nucleotide mismatches with the target sequence, and the method comprises a step of expressing in cells:
(a) a crRNA containing a spacer sequence targeting the target sequence and the mismatch sequence; and
(b) a CasΦ protein derived from a huge phage and able to cleave the target sequence and mismatch sequence.

2. The genome editing method according to claim 1, wherein the CasΦ protein is at least one protein selected from the group consisting of CasΦ1, CasΦ2, vCasΦ and CasΦ3.

3. The genome editing method according to claim 1, wherein the CasΦ protein includes an amino acid sequence having at least 80% sequence identity with an amino acid sequence selected from the group consisting of SEQ ID NO: 1, 2, 3 and 4.

4. The genome editing method according to claim 1, wherein the duplicated genes are duplicated sequences with functional redundancy.

5. The genome editing method according to claim 1, wherein the duplicated genes include a PAM sequence near the target sequence.

6. The genome editing method according to claim 1, wherein the spacer sequence is determined so as to completely match the target sequence.

7. The genome editing method according to claim 1, wherein the duplicated genes have only a target sequence with a nucleotide length of 16 or greater, or a target sequence with a nucleotide length of 16 or greater and a mismatch sequence containing 1 to 2 nucleotide mismatches with the target sequence.

8. The genome editing method according to claim 1, wherein the duplicated genes have a target sequence with a nucleotide length of 17 or greater and a mismatch sequence containing 1 to 2 nucleotide mismatches with the target sequence.

9. The genome editing method according to claim 1, wherein the duplicated genes are present in 2 to 100 copies in the genome of the cell.

10. The genome editing method according to claim 9, wherein all or some of the duplicated genes are edited, among the duplicated genes in the genome of the cell.

11. The genome editing method according to claim 1, wherein the cells are plant cells.

12. The genome editing method according to claim 1, wherein the crRNA and/or the CasΦ protein are expressed by being introduced into the cell using an expression vector.

13. The genome editing method according to claim 12, wherein the expression vector is a transient or a constitutive expression vector.

14. A kit for genome editing of duplicated genes in a cell, wherein the duplicated genes have only a 16-nucleotide length target sequence for genome editing, or a 16- or 17-nucleotide length target sequence and a mismatch sequence containing 1 to 2 nucleotide mismatches with the target sequence, and the kit comprises:
(a) an expression cassette for crRNA which is able to transfer a spacer sequence targeting the target sequence and the mismatch sequence; and
(b) an expression cassette for a CasΦ protein derived from a huge phage and able to cleave the target sequence and the mismatch sequence.
